SEQUENCE LISTING (16 sequences)

SEQ ID NO: 1 - protein, 87 aa, Homo sapiens
MTMYLWLKLLAFGFAFLDTEVFVTGQSPTPSPTGHLQAEEQGSQSKSPNLKSREADSSAFSWWPKAREPLTNHWSKSKSPKAEELGV

SEQ ID NO: 2 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 3 - protein, 1145 aa, Homo sapiens
MTMYLWLKLLAFGFAFLDTEVFVTGQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 4 - protein, 373 aa, Homo sapiens
MPFGIRMLLRAHKPGRSEMTTSLDTVETFGTTSYYDDVGLLCEKADTRALMAQFVPPLYSLVFTVGLLGNVVVVMILIKYRRLRIMTNIYLLNLAISDLLFLVTLPFWIHYVRGHNWVFGHGMCKLLSGFYHTGLYSEIFFIILLTIDRYLAIVHAVFALRARTVTFGVITSIVTWGLAVLAALPEFIFYETEELFEETLCSALYPEDTVYSWRHFHTLRMTIFCLVLPLLVMAICYTGIIKTLLRCPSKKKYKAIRLIFVIMAVFFIFWTPYNVAILLSSYQSILFGNDCERSKHLDLVMLVTEVIAYSHCCMNPVIYAFVGERFRKYLRHFFHRHLLMHLGRYIPFLPSEKLERTSSVSPSTAEPELSIVF

SEQ ID NO: 5 - protein, 355 aa, Homo sapiens
MTTSLDTVETFGTTSYYDDVGLLCEKADTRALMAQFVPPLYSLVFTVGLLGNVVVVMILIKYRRLRIMTNIYLLNLAISDLLFLVTLPFWIHYVRGHNWVFGHGMCKLLSGFYHTGLYSEIFFIILLTIDRYLAIVHAVFALRARTVTFGVITSIVTWGLAVLAALPEFIFYETEELFEETLCSALYPEDTVYSWRHFHTLRMTIFCLVLPLLVMAICYTGIIKTLLRCPSKKKYKAIRLIFVIMAVFFIFWTPYNVAILLSSYQSILFGNDCERSKHLDLVMLVTEVIAYSHCCMNPVIYAFVGERFRKYLRHFFHRHLLMHLGRYIPFLPSEKLERTSSVSPSTAEPELSIVF

SEQ ID NO: 6 - protein, 376 aa, Homo sapiens
MPFGIRMLLRAHKPGSSRRSEMTTSLDTVETFGTTSYYDDVGLLCEKADTRALMAQFVPPLYSLVFTVGLLGNVVVVMILIKYRRLRIMTNIYLLNLAISDLLFLVTLPFWIHYVRGHNWVFGHGMCKLLSGFYHTGLYSEIFFIILLTIDRYLAIVHAVFALRARTVTFGVITSIVTWGLAVLAALPEFIFYETEELFEETLCSALYPEDTVYSWRHFHTLRMTIFCLVLPLLVMAICYTGIIKTLLRCPSKKKYKAIRLIFVIMAVFFIFWTPYNVAILLSSYQSILFGNDCERSKHLDLVMLVTEVIAYSHCCMNPVIYAFVGERFRKYLRHFFHRHLLMHLGRYIPFLPSEKLERTSSVSPSTAEPELSIVF

SEQ ID NO: 7 - protein, 355 aa, Homo sapiens; identical to SEQ ID NO: 5

SEQ ID NO: 8 - protein, 290 aa, Homo sapiens
MGGGAGERLFTSSCLVGLVPLGLRISLVTCPLQCGIMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 9 - protein, 289 aa, Homo sapiens
MGGGAGERLFTSSCLVGLVPLGLRISLVTCPLQCGIMWQLLLPTALLLLVSAGMRTDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 10 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 11 - protein, 254 aa, Homo sapiens; identical to SEQ ID NO: 10

SEQ ID NO: 12 - protein, 253 aa, Homo sapiens
MWQLLLPTALLLLVSAGMRTDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 13 - protein, 233 aa, Homo sapiens
MWQLLLPTALLLLVSAGMRTEDLPKAVVFLEPQWYSVLEKDSVTLKCQGAYSPEDNSTQWFHNENLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 14 - protein, 269 aa, Homo sapiens
MGGGTGERLFTPSCLVGLVPLGLRISLVTCPLQCGIMWQLLLPTALLLLVSAGMRTEDLPKAVVFLEPQWYSVLEKDSVTLKCQGAYSPEDNSTQWFHNENLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 15 - protein, 788 aa, Homo sapiens
MRARPRPRPLWATVLALGALAGVGVGGPNICTTRGVSSCQQCLAVSPMCAWCSDEALPLGSPRCDLKENLLKDNCAPESIEFPVSEARVLEDRPLSDKGSGDSSQVTQVSPQRIALRLRPDDSKNFSIQVRQVEDYPVDIYYLMDLSYSMKDDLWSIQNLGTKLATQMRKLTSNLRIGFGAFVDKPVSPYMYISPPEALENPCYDMKTTCLPMFGYKHVLTLTDQVTRFNEEVKKQSVSRNRDAPEGGFDAIMQATVCDEKIGWRNDASHLLVFTTDAKTHIALDGRLAGIVQPNDGQCHVGSDNHYSASTTMDYPSLGLMTEKLSQKNINLIFAVTENVVNLYQNYSELIPGTTVGVLSMDSSNVLQLIVDAYGKIRSKVELEVRDLPEELSLSFNATCLNNEVIPGLKSCMGLKIGDTVSFSIEAKVRGCPQEKEKSFTIKPVGFKDSLIVQVTFDCDCACQAQAEPNSHRCNNGNGTFECGVCRCGPGWLGSQCECSEEDYRPSQQDECSPREGQPVCSQRGECLCGQCVCHSSDFGKITGKYCECDDFSCVRYKGEMCSGHGQCSCGDCLCDSDWTGYYCNCTTRTDTCMSSNGLLCSGRGKCECGSCVCIQPGSYGDTCEKCPTCPDACTFKKECVECKKFDRGALHDENTCNRYCRDEIESVKELKDTGKDAVNCTYKNEDDCVVRFQYYEDSSGKSILYVVEEPECPKGPDILVVLLSVMGAILLIGLAALLIWKLLITIHDRKEFAKFEEERARAKWDTANNPLYKEATSTFTNITYRGT

SEQ ID NO: 16 - protein, 1039 aa, Homo sapiens
MARALCPLQALWLLEWVLLLLGPCAAPPAWALNLDPVQLTFYAGPNGSQFGFSLDFHKDSHGRVAIVVGAPRTLGPSQEETGGVFLCPWRAEGGQCPSLLFDLRDETRNVGSQTLQTFKARQGLGASVVSWSDVIVACAPWQHWNVLEKTEEAEKTPVGSCFLAQPESGRRAEYSPCRGNTLSRIYVENDFSWDKRYCEAGFSSVVTQAGELVLGAPGGYYFLGLLAQAPVADIFSSYRPGILLWHVSSQSLSFDSSNPEYFDGYWGYSVAVGEFDGDLNTTEYVVGAPTWSWTLGAVEILDSYYQRLHRLRGEQMASYFGHSVAVTDVNGDGRHDLLVGAPLYMESRADRKLAEVGRVYLFLQPRGPHALGAPSLLLTGTQLYGRFGSAIAPLGDLDRDGYNDIAVAAPYGGPSGRGQVLVFLGQSEGLRSRPSQVLDSPFPTGSAFGFSLRGAVDIDDNGYPDLIVGAYGANQVAVYRAQPVVKASVQLLVQDSLNPAVKSCVLPQTKTPVSCFNIQMCVGATGHNIPQKLSLNAELQLDRQKPRQGRRVLLLGSQQAGTTLNLDLGGKHSPICHTTMAFLRDEADFRDKLSPIVLSLNVSLPPTEAGMAPAVVLHGDTHVQEQTRIVLDCGEDDVCVPQLQLTASVTGSPLLVGADNVLELQMDAANEGEGAYEAELAVHLPQGAHYMRALSNVEGFERLICNQKKENETRVVLCELGNPMKKNAQIGIAMLVSVGNLEEAGESVSFQLQIRSKNSQNPNSKIVLLDVPVRAEAQVELRGNSFPASLVVAAEEGEREQNSLDSWGPKVEHTYELHNNGPGTVNGLHLSIHLPGQSQPSDLLYILDIQPQGGLQCFPQPPVNPLKVDWGLPIPSPSPIHPAHHKRDRRQIFLPEPEQPSRLQDPVLVSCDSAPCTVVQCDLQEMARGQRAMVTVLAFLWLPSLYQRPLDQFVLQSHAWFNVSSLPYAVPPLSLPRGEAQVWTQLLRALEERAIPIWWVLVGVLGGLLLLTILVLAMWKVGFFKRNRPPLEEDDEEGE

(19) United States
(12) Patent Application Publication
(10) Pub. No.: US 2015/0253327 A1
(43) Pub. Date: Sep. 10, 2015

(54) DIAGNOSIS AND TREATMENT OF ASPIRIN-EXACERBATED RESPIRATORY DISEASE (AERD)

(71) Applicant: BRIGHAM AND WOMEN'S HOSPITAL, INC., Boston, MA (US)

(72) Inventors: Tanya M. Laidlaw, Needham, MA (US); Joshua Boyce, Boston, MA (US)

(73) Assignee: BRIGHAM AND WOMEN'S HOSPITAL, INC., Boston, MA (US)

(21) Appl. No.: 14/425,454

(22) PCT Filed: Aug. 15, 2013
(86) PCT No.: PCT/US2013/055049

§ 371 Date: Mar. 3, 2015

Related U.S. Application Data
(60) Provisional application No. 61/698,058, filed on Sep. 7, 2012.

(51) Int. Cl. G01N 33/569

(52) U.S. Cl. CPC G01N 33/56972; G01N 2333/70557; G01N 2333/70589; G01N 2333/70535; G01N 2800/12

(57) ABSTRACT

Described herein are assays and methods relating to the diagnosis, prognosis, and treatment of aspirin-exacerbated respiratory disease (AERD) by detecting and/or measuring the level of platelet-bound leukocytes in a subject or a sample obtained from a subject.

### CROSS-REFERENCE TO RELATED APPLICATIONS

This application claims benefit under 35 U.S.C. §119(e) of U.S. Provisional Application No. 61/698,058 filed Sep. 7, 2012, the contents of which are incorporated herein by reference in their entirety.

### GOVERNMENT SUPPORT

This invention was made with federal funding under Grant Nos. U 19 AI095219-01, AI078908, AT002782, and AI082369 awarded by the National Institutes of Health. The U.S. government has certain rights in the invention.

### SEQUENCE LISTING

The instant application contains a Sequence Listing which has been submitted in ASCII format via EFS-Web and is hereby incorporated by reference in its entirety. Said ASCII copy, created on Aug. 14, 2013, is named 043214-074911-PCT_SEtxt and is 66,774 bytes in size.

### TECHNICAL FIELD

The technology described herein relates to the diagnosis and treatment of aspirin-exacerbated respiratory disease (AERD).

### BACKGROUND

Aspirin-exacerbated respiratory disease (AERD) is a severe disease characterized clinically by adult onset asthma, severe eosinophilic rhinosinusitis, and recurrent nasal polyposis. Current diagnosis relies on identifying bronchoconstriction in response to a challenge with aspirin, a procedure that is risky and that community-based physicians are generally reluctant to carry out. Identification of individuals with AERD is critical because the prognosis and treatment options are different from aspirin tolerant asthma. Moreover, counseling patients to avoid nonsteroidal anti-inflammatory drugs is also essential for their safety. There is a need for safer and more effective methods and assays for diagnosis of AERD, as well as in vitro diagnosis assays which can easily be utilized by community-based physicians.

### SUMMARY

The technology described herein is generally directed to diagnostic methods, assays, and systems, as well as methods of treatment, for AERD. As described in the Examples herein, the inventors have discovered that the leukocytes (e.g. white blood cells) of subjects with AERD are more likely to be bound to platelets (a type of blood cell involved in the development of blood clots), as compared to control subjects without AERD. Thus, the level of leukocytes which are specifically bound to, or can bind to, platelets (i.e. platelet-adherent leukocytes) can be used as the basis of diagnostic methods, assays, and systems, as well as methods of treatment, for AERD.

In some embodiments, the level of platelet-adherent leukocytes in a sample can be determined by taking advantage of polypeptides present on cell surfaces which are specific to certain cell types (i.e. cell type-specific markers). For example, platelets, but not other cell types, express the polypeptides CD61 and CD41 on their cell surfaces. Thus, if CD61 and/or CD41 is present on the surface of a cell, that cell is a platelet, i.e. cells which are CD61⁺ and/or CD41⁺ are platelets. Likewise, there are markers which are specific for leukocytes generally (e.g. CD45) or for subpopulations of leukocytes (e.g. CCR3 is a marker specific for eosinophils and CD16 is a marker specific for neutrophils). Thus, if CD45 is present on the surface of a cell, that cell is a leukocyte, i.e. cells which are CD45⁺ are leukocytes. These markers can be detected using labeled antibody reagents.

Accordingly, in some embodiments of the technology described herein, a sample obtained from a subject can be contacted with a platelet-specific antibody reagent (e.g. an antibody-reagent specific for CD61 or CD41) and a leukocyte-specific antibody reagent (e.g. an antibody-reagent specific for CD45). If a platelet-specific antibody reagent and a leukocyte-specific antibody reagent both bind to the same location (or group of cells), it can indicate the presence of a platelet-adherent leukocyte. Conversely, if a leukocyte-specific antibody binds to a particular location (or group of cells), but the platelet-specific antibody reagent does not bind to the same location (or group of cells), then it can indicate the presence of a leukocyte which is not bound to a platelet. The results of such assays, and assays based on the same principle, can be used to determine the level of platelet-adherent leukocytes in a sample obtained from a subject. Such determinations can comprise one step of diagnosing or treating AERD according to some embodiments of the invention as described herein.

In one aspect, the technology described herein relates to an assay comprising: contacting a test sample from a subject with a platelet-specific reagent; measuring the percentage of leukocytes to which the reagent is bound; wherein the binding of the platelet-specific reagent to a leukocyte indicates the presence of a platelet bound to the leukocyte; wherein an increased level of platelet-bound leukocytes relative to a reference level indicates the subject has an increased risk of having aspirin-exacerbated respiratory disease (AERD).

In one aspect, the technology described herein relates to an assay comprising: (a) contacting the a test sample comprising leukocytes from the peripheral blood of a subject with a platelet-specific antibody reagent and a leukocyte-specific antibody reagent; and (b) detecting the presence or intensity of a detectable signal associated with individual cells of the sample using flow cytometry; wherein the antibody reagents comprise a detectable label or a means of generating a detectable signal; wherein the binding of the platelet-specific antibody reagent to a leukocyte indicates the presence of a platelet-bound leukocyte; wherein the binding of the platelet-specific antibody reagent to a leukocyte is determined by the colocalization and/or concurrent detection of the platelet-specific antibody reagent and the leukocyte-specific antibody reagent; and wherein an increased level platelet-bound leukocytes within the population of leukocytes, as indicated by the detectable signals, relative to a reference level indicates the subject has an increased risk of having aspirin-exacerbated respiratory disease (AERD).

In one aspect, the technology described herein relates to an assay to determine if a subject with a respiratory disease will benefit from treatment with an aspirin-exacerbated respiratory disease (AERD) therapy selected from the group consisting of: aspirin desensitization and high-dose aspirin therapy; a P2Y12 inhibitor; montelukast; a thromboxane receptor antagonist; a 5-lipoxygenase inhibitor; and zileuton; the assay comprising: contacting a test sample obtained from the subject with a platelet-specific reagent; measuring the percentage of leukocytes to which the reagent is bound; wherein the binding of the platelet-specific reagent to a leukocyte indicates the presence of a platelet bound to the leukocyte; wherein the binding of the platelet-specific antibody reagent to a leukocyte is determined by the colocalization and/or concurrent detection of the platelet-specific antibody reagent and the leukocyte-specific antibody reagent; and wherein an increased level of platelet-bound leukocytes relative to a reference level indicates the subject will benefit from treatment with an AERD therapy.

In one aspect, the technology described herein relates to an assay to determine if a subject with a respiratory disease should not be administered a cyclooxygenase-1 (COX1) inhibitor. the assay comprising: contacting a test sample obtained from the subject with a platelet-specific reagent; measuring the percentage of leukocytes to which the reagent is bound; wherein the binding of the platelet-specific reagent to a leukocyte indicates the presence of a platelet bound to the leukocyte; and wherein an increased level of platelet-bound leukocytes relative to a reference level indicates the subject should not be administered a COX1 inhibitor. In some embodiments, the cyclooxygenase-1 (COX1) inhibitor can be selected from the group consisting of: aspirin; diclofenac; ibuprofen; naproxen; mefenamic acid; indomethacin; ketoprofen; piroxicam; diflunisal; salsalate; dexibuprofen; fenoprofen; dexketoprofen; flurbiprofen; oxaprozin; loxoprofen; indomethacin; sulindac; etodolac; ketorolac; nabumetone; meloxicam; tenoxicam; droxicam; lornoxicam; isoxicam; mefenamic acid; meclofenamic acid; flufenamic acid; and tolfenamic acid.

In one aspect, the technology described herein can relate to a method of administering a treatment for a subject with a respiratory disease, the method comprising: contacting a test sample from the subject with a platelet-specific antibody reagent; measuring the percentage of leukocytes to which the reagent is bound; wherein the binding of the reagent to a leukocyte indicates that the leukocyte is a platelet-bound leukocyte; and administering a therapy for aspirin-exacerbated respiratory disease (AERD) if the level of platelet-bound leukocytes is increased relative to a reference level; wherein the therapy for AERD is selected from the group consisting of: aspirin desensitization and high-dose aspirin therapy; a P2Y12 inhibitor; montelukast; a thromboxane receptor antagonist; a 5-lipoxygenase inhibitor; and zileuton.

In one aspect, the technology described herein can relate to a method of identifying a subject with a respiratory disease who will benefit from treatment a therapy for aspirin-exacerbated respiratory disease (AERD), the method comprising: contacting a test sample from the subject with a platelet-specific antibody reagent; and measuring the percentage of leukocytes to which the reagent is bound; wherein the binding of the reagent to a leukocyte indicates that the leukocyte is a platelet-bound leukocyte; wherein the subject is identified as needing treatment with a therapy for AERD if the level of platelet-bound leukocytes is increased relative to a reference level; wherein the therapy for AERD is selected from the group consisting of: aspirin desensitization and high-dose aspirin therapy; a P2Y12 inhibitor; montelukast; a thromboxane receptor antagonist; a 5-lipoxygenase inhibitor; and zileuton.

In one aspect, the technology described herein can relate to a method of determining if a subject is at increased risk of having aspirin-exacerbated respiratory disease (AERD), the method comprising: measuring the percentage of platelet-bound leukocytes in a sample obtained from the subject; wherein an increased level of platelet-bound leukocytes relative to a reference level indicates the subject is at increased risk of having AERD.

In one aspect, the technology described herein can relate to a computer system for determining if subject has an increased risk of having aspirin-exacerbated respiratory disease (AERD), the system comprising: a measuring module configured to measure the level of platelet-bound leukocytes in a population of leukocytes in a test sample obtained from a subject; a storage module configured to store output data from the measuring module; a comparison module adapted to compare the data stored on the storage module with a reference level, and to provide a retrieved content, and a display module for displaying whether the level of platelet-bound leukocytes in a population of leukocytes in a test sample obtained from a subject is greater, by a statistically significant amount, than the reference level and/or displaying the relative levels of platelet-bound leukocytes in a population of leukocytes. In some embodiments, the measuring module can measure the presence or intensity of a detectable signal from an immunoassay indicating the presence of platelet-specific antibody reagent on the cells in the test sample. In some embodiments, if the computing module determines that the level of platelet-bound leukocytes in a population of leukocytes in the test sample obtained from a subject is greater by a statistically significant amount than the reference level, the display module can display a signal indicating that the level in the sample obtained from a subject is greater than that of the reference level. In some embodiments, the signal can indicate that the subject is at increased risk of having aspirin-exacerbated respiratory disease (AERD). In some embodiments, the signal can indicate the subject can benefit from treatment with a wherein the therapy for AERD is selected from the group consisting of: aspirin desensitization and high-dose aspirin therapy; a P2Y12 inhibitor; montelukast; a thromboxane receptor antagonist; a 5-lipoxygenase inhibitor; and zileuton. In some embodiments, the signal can indicate the subject should not be administered a cyclooxygenase-1 (COX1) inhibitor. In some embodiments, the signal can indicate the degree to which the level of platelet-bound leukocytes in a population of leukocytes in the sample obtained from the subject vary from the reference level.

In some embodiments of any of the foregoing aspects, the platelet-specific reagent can be selected from the group consisting of: a CD61-binding reagent and a CD41-binding reagent. In some embodiments of any of the foregoing aspects, the platelets can be CD61+ cells. In some embodiments of any of the foregoing aspects the platelets can be CD41+ cells. In some embodiments of any of the foregoing aspects the leukocytes can be CD45⁺ cells. In some embodiments of any of the foregoing aspects, the leukocyte-specific antibody reagent can be an anti-CD45 antibody reagent. In some embodiments of any of the foregoing aspects, the leukocytes can be neutrophils. In some embodiments of any of the foregoing aspects, the neutrophils can be CD16⁺ cells. In some embodiments of any of the foregoing aspects, the leukocytes can be eosinophils. In some embodiments of any of the foregoing aspects, the eosinophils can be CCR3⁺ cells.

In some embodiments of any of the foregoing aspects, the sample can comprise a biological tissue selected from the group consisting of: whole blood; peripheral blood; whole peripheral blood; a nasal polyp; and products thereof.

In some embodiments of any of the foregoing aspects, the level of platelet-bound leukocytes can be measured by flow cytometry. In some embodiments of any of the foregoing aspects, the level of platelet-bound leukocytes can be measured by immunocytological methods. In some embodiments of any of the foregoing aspects, the platelet-specific antibody reagent can comprise an antibody or a polypeptide comprising an antigen-binding domain of an antibody.

In some embodiments of any of the foregoing aspects, the subject can have increased level of platelet-bound leukocytes relative to a reference level if the level of platelet-bound leukocytes is at least 2× that of the reference level. In some embodiments of any of the foregoing aspects, the subject can have an increased level of platelet-bound leukocytes relative to a reference level if the level of platelet-bound leukocytes is at least 3× that of the reference level. In some embodiments of any of the foregoing aspects, the subject can have an increased level of platelet-bound leukocytes relative to a reference level if at least 15% of the leukocytes are platelet-bound leukocytes. In some embodiments of any of the foregoing aspects, the subject can have an increased level of platelet-bound leukocytes relative to a reference level if at least 50% of the eosinophils are platelet-bound eosinophils. In some embodiments of any of the foregoing aspects, the subject can have an increased level of platelet-bound leukocytes relative to a reference level if at least 25% of the neutrophils are platelet-bound neutrophils.

In some embodiments of any of the foregoing aspects, the reference level of platelet-bound leukocytes can be the level of platelet-bound leukocytes in a healthy subject without a respiratory disease. In some embodiments of any of the foregoing aspects, the reference level of platelet-bound leukocytes can be the level of platelet-bound leukocytes in a subject with aspirin-tolerant asthma.

In some embodiments of any of the foregoing aspects, the subject can be a human. In some embodiments of any of the foregoing aspects, the method, system, or assay can further comprise creating a report based on the level of platelet-bound leukocytes.

In one aspect, described herein is a method of directing and/or monitoring the treatment of a subject in need of treatment for AERD, the method comprising a) measuring a first level of platelet-bound leukocytes in a sample obtained from a subject as described herein; b) administering a treatment for AERD if the subject is determined to have an increased level of platelet-bound leukocytes relative to a reference; c) measuring a second level of platelet-bound leukocytes in a sample obtained from a subject as described herein; and d) adjusting the dosage of the treatment as indicated by the second level of platelet-bound leukocytes. In some embodiments, the treatment can be selected from the group consisting of a P2Y12 inhibitor; a leukotriene receptor antagonist; montelukast; a thromboxane receptor antagonist; a 5-lipoxygenase inhibitor; and zileuton.

### BRIEF DESCRIPTION OF THE DRAWINGS

FIGS. 1A-1B depict the detection of platelet-leukocyte aggregates in nasal polyp tissue. FIG. 1A depicts a graph of the total numbers of CD45+ cells that colocalized with CD61. FIG. 1B depicts a graph of the percentages of CD454-cells that colocalized with CD61 in the nasal polyp tissue from aspirin-tolerant controls with sinusitis (n=4) and subjects with AERD (n=6). Data are expressed as mean±SD.

FIGS. 2A-2B depict the results of experiments demonstrating that platelet-adherent leukocytes are identifiable in peripheral blood. FIG. 2A depicts representative histograms of platelet-adherent eosinophils (identified as CCR3+CD45+ cells in the granulocyte side scatter [SSC] gate), neutrophils (CD16+CD45+ cells in the granulocyte SSC gate), monocytes (CD45+ in the monocyte SSC gate), and lymphocytes (CD45+ cells in lymphocyte SSC gate) in blood from an ATA control (top) and a subject with AERD (bottom). The percentages of each cell type with adherent platelets are shown. FIG. 2B depicts a graph of the percentages of leukocytes with adherent platelets (as determined by staining with CD61) in blood of nonasthmatic controls (n=9), ATA controls (n=13), and subjects with AERD (n=15). Data are expressed as mean±SD (stars indicate P=0.001).

FIGS. 3A-3D depict the results of experiments demonstrating expression of integrins by platelet-adherent and -nonadherent leukocyte subsets. FIG. 3A depicts representative histograms of relative CD18 expression by CD61− platelet-nonadherent (solid gray) and CD61+ platelet-adherent (black line) peripheral blood eosinophils (top), neutrophils (middle), and monocytes (bottom) are shown for a subject with AERD. FIGS. 3B-3D depict graphs of the relative expression of CD18 and CD49 on eosinophils (FIG. 3B), CD18 on neutrophils (FIG. 3C), and CD18 and CD11b on monocytes (FIG. 3D), comparing the platelet-adherent and platelet-nonadherent leukocyte subsets in nonasthmatic controls (n=7), ATA controls (n=10), and subjects with AERD (n=9). MFI indicates mean fluorescence intensity. Platelet-free CD61− leukocyte subsets are shown in white columns, and CD61+ leukocyte subsets are shown in hatched columns. Data are expressed as mean+SEM (one star indicates P<0.05, two stars indicate P<0.01, 3 stars indicate P<0.001).

FIGS. 4A-4D depict the results of experiments demonstrating the contribution of platelet LTC4S to cysLT production by peripheral blood granulocytes. FIG. 4A depicts Western blot analysis of platelets for LTC4S protein in 3 ATA controls and 4 subjects with AERD. In FIG. 4B the removal of adherent platelets by trypsinization is shown cytofluorographically for a subject with AERD. Isolated granulocytes were stained for CD45 and analyzed for their expression of CD61 before trypsinization (black line) and again after trypsinization (solid gray). FIG. 4C depicts the conversion of LTA4-ME to LTC4-ME by washed platelets (left section of panel) and by granulocytes (right section of panel) without and with trypsinization to remove adherent platelets from subjects with ATA (gray columns; n=6) and AERD (black columns; n=7). FIG. 4D demonstrates the A23187-induced production of LTC4 (top) and the sum of all 5-LO pathway products [LTB4,LTC4,LTD4, (5,6)-dihydroxy-7,9trans-11,14-cis-eicosatetraenoic acid [5,6-DiHETE], and 6-trans-LT84; bottom] by granulocytes without and without trypsinization to remove adherent platelets from subjects with ATA (n=8) and AERD (n=10). Data in FIGS. 4C and 4D are expressed as mean+SEM (one star indicates P<0.05, two stars indicate P<0.01).

FIGS. 5A-5B depict the results of experiments demonstrating that platelet-adherent leukocytes correlate with systemic cysLT production. FIG. 5A depicts baseline urinary LTE4 (top) and TXB2 (bottom) levels analyzed by gas chromatography-mass spectrometry from nonasthmatic controls (n=8), ATA controls (n=9) and subjects with AERD (n=10). Data are expressed as mean+SD (three stars indicate P<0.001). FIG. 5B depicts baseline urinary LTE4 levels plotted against the corresponding percentages of platelet-adherent eosinophils (top), neutrophils (middle), and monocytes (bottom) in the peripheral blood of each subject. Effect size, determined with Pearson correlation coefficient, is denoted as an r value displayed for each cell type. White circles, nonasthmatic controls; gray squares, ATA controls; black triangles, AERD subjects.

FIG. 6 depicts representative cytofluorographic identification of peripheral blood leukocytes in a subject with AERD. Side scatter characteristics and relative expression of CD45 allows for identification of granulocyte, monocyte, and lymphocyte populations (bottom panel). Two distinct populations within the granulocyte gate are further defined as CD16+ neutrophils or CCR3+ eosinophils (top panels).

FIGS. 7A-7C demonstrate expression of integrins by platelet-adherent and -nonadherent leukocyte subsets, and constitutive PSGL-1 expression by leukocytes. FIGS. 7A-7B depict the relative expression of CD11a on eosinophils (FIG. 7A) and CD11a and CD49d on monocytes (FIG. 7B), comparing the platelet-adherent and platelet-nonadherent leukocyte subsets in nonasthmatic controls (n=7), ATA controls (n=10), and subjects with AERD (n=9). Platelet-free CD61− leukocyte subsets are shown in white columns, CD61+ leukocyte subsets are shown in hatched columns. (one star=P<0.05, two stars=P<0.01, three stars=P<0.001). Data are expressed as mean+SEM. FIG. 7C demonstrates constitutive expression of PSGL-1 by peripheral blood leukocytes from nonasthmatic controls (n=7), ATA controls (n=7), and subjects with AERD (n=8). Data are expressed as mean+SD.

FIG. 8 demonstrates that trypsinization does not compromise cell functionality. Generation of cysLTs (top panel) and all 5-LO pathway products (LTB₄, LTC₄, LTD₄, 5,6-DiHETE, and 6-trans-LTB₄) (bottom panel) by A23187-stimulated granulocytes with and without trypsinization to remove adherent platelets, and after the addition of 200×10⁶ autologous platelets. Data are from 6 subjects with AERD, expressed as mean+SEM. The effect of adding autologous platelets to trypsinized granulocytes was significant (P=0.01).

FIG. 9 is a diagram of an exemplary embodiment of a system for performing an assay for determining the level of platelet-adherent leukocytes in sample obtained from a subject.

FIG. 10 is a diagram of an embodiment of a comparison module as described herein.

FIG. 11 is a diagram of an exemplary embodiment of an operating system and instructions for a computing system as described herein.

FIGS. 12A-12C demonstrate the relationships between platelet-adherent neutrophils, LTB₄ generation, and suppression of 5-LO activity in AERD. FIG. 12A depicts a graph of percentages of platelet-adherent neutrophils (determined by CD61⁺ expression) in whole blood from eight subjects with AERD plotted against quantity of LTB₄ generated by fMLP-stimulated granulocytes from the same individuals. Percent suppression of fMLP-induced LTB₄ by pretreatment with (FIG. 12B) PGE₂ or (FIG. 12C) the EP₂ receptor-specific agonist was plotted against percentages of platelet-adherent neutrophils in the peripheral blood of each subject. Effect size, determined with Pearson correlation coefficient, is denoted as an r value.

### DETAILED DESCRIPTION

Embodiments of the technology described herein relate to methods based upon the inventors' discovery that subjects with aspirin-exacerbated respiratory disease (AERD) have higher levels of platelet-adherent leukocytes (i.e. white blood cells which are specifically bound to or can specifically bind to platelets) as compared to subjects without AERD. Described herein are methods of diagnosis, prognosis, and treatment for AERD as well as assays, systems, and kits relating thereto.

In some embodiments, the level of platelet-adherent leukocytes in a sample can be determined by taking advantage of cell-type specific markers present on cell surfaces. For example, CD41 and CD61 are platelet-specific markers while CD45 is a leukocyte-specific marker (CCR3 and CD16 are specific for subpopulations of leukocytes; eosinophils and neutrophils, respectively). Detectably labeled antibody reagents specific for these markers can be used as described herein. Briefly, if a platelet-specific antibody reagent and leukocyte-specific antibody reagent colocalize, it indicates the presence of a platelet-adherent leukocyte. In some embodiments, platelet-adherent leukocytes can bind platelets, e.g. CD61+ platelets. Accordingly, in some embodiments, a platelet-adherent leukocyte can be present within a group of cells (e.g. a leukocyte and one or more platelets) which will be a CD45+/CD61+ group of cells. Detection of colocalization of a platelet-specific antibody reagent and a leukocyte-specific antibody reagent (e.g. antibody reagents specific for CD61 and CD45, respectively) can indicate the presence of a platelet-adherent leukocyte. Described herein are assays, methods, and systems, as well as methods of treatment, for AERD which relate to the level of platelet-adherent leukocytes being higher in subjects having, or at risk of having, AERD.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein generally to mean a decrease by a statistically significant amount. However, for avoidance of doubt, “reduced”, “reduction”, “decrease”, or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level or non-detectable level as compared to a reference level), or any decrease between 10-100% as compared to a reference level. In the context of a marker or symptom is meant a statistically significant decrease in such level. The decrease can be, for example, at least 10%, at least 20%, at least 30%, at least 40% or more, and is preferably down to a level accepted as within the range of normal for an individual without such disorder.

The terms “increased”, “increase”, “enhance”, or “activate” are all used herein to generally mean an increase by a statically significant amount; for the avoidance of doubt, the terms “increased”, “increase”, “enhance”, or “activate” mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom is meant a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of AERD. A subject can be male or female.

A subject can be one who has been previously diagnosed with or identified as suffering from or having a condition in need of treatment (e.g. a respiratory disease, asthma, and/or AERD) or one or more complications related to such a condition, and optionally, have already undergone treatment for, e.g., AERD or the one or more complications related to AERD.

Symptoms of AERD can include, but are not limited to, chronic and/or non-allergic rhinitis, nasal polyps, and/or asthma, and the precipitation of both asthma and/or rhinitis attacks after ingestion of aspirin or other COX-1 inhibitors. Alternatively, a subject can also be one who has not been previously diagnosed as having, e.g., AERD or one or more complications related to AERD. For example, a subject can be one who exhibits one or more risk factors for, e.g., AERD or one or more complications related to AERD or a subject who does not exhibit risk factors. In some embodiments, a subject can be one with a respiratory disorder, i.e. a perturbation or impediment affecting the respiratory system and having any etiology or cause. In some embodiments, a subject can be one with asthma. As used herein, asthma refers to a chronic inflammatory disease of the respiratory system in which the airway occasionally constricts, becomes inflamed, and is lined with excessive amounts of mucus, often in response to one or more triggers. Asthma can also include a reversible airway obstruction in an individual over a period of time. Asthma can be allergic/atopic or non-allergic. Asthma is characterized by the presence of cells such as eosinophils, mast cells, basophils, and activated T lymphocytes in the airway walls. Asthma can worsen over time, leading to thickening of basement membranes and fibrosis. Asthma can be characterized by increased airway hyper responsiveness to a variety of stimuli, and airway inflammation and constriction/narrowing. This airway constriction/narrowing causes symptoms such as wheezing, shortness of breath, chest tightness, and coughing. The airway constriction responds to bronchodilators. Between episodes, most patients feel well but can have mild symptoms and they can remain short of breath after exercise for longer periods of time than the unaffected individual. The symptoms of asthma can range from mild to life threatening.

A “subject in need” of treatment for a particular condition can be a subject having that condition, diagnosed as having that condition, or at risk of developing that condition.

As used herein, the term “platelet” refers to a nonnucleated, disklike cell found in mammalian blood plasma and which promotes clotting. In some embodiments, a platelet can be a CD61+ cell. In some embodiments, a platelet can be a CD61+ cell which is present in blood or a blood sample. In some embodiments, a platelet can be a CD41+ cell. In some embodiments, a platelet can be a CD41+ cell which is present in blood or a blood sample.

As used herein, the term “leukocyte” refers to a white blood cell that plays a role in the immune system and includes granulocytes (e.g. basophils, eosinophils, and neutrophils), lymphocytes, macrophages, dendritic cells, mast cells, NK cells, and monocytes.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments (see, e.g. de Wildt et al., Eur J. Immunol 1996; 26(3):629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The terms “antigen-binding fragment” or “antigen-binding domain”, which are used interchangeable herein are used herein to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., U.S. Pat. Nos. 5,260,203, 4,946,778, and 4,881,175; Bird et al. (1988) Science 242:423-426; and Huston et al. (1988) Proc. Natl. Acad. Sci. USA 85:5879-5883. Antibody fragments can be obtained using any appropriate technique including conventional techniques known to those of skill in the art. The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition, irrespective of how the antibody was generated.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity.

The term “label” refers to a composition capable of producing a detectable signal indicative of the presence of an antibody reagent (e.g. a bound antibody reagent). Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

As used herein, the term “proteins” and “polypeptides” are used interchangeably herein to designate a series of amino acid residues connected to the other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. a respiratory disease, asthma, and/or AERD. The term “treating” includes reducing or alleviating at least one adverse effect or symptom of a condition, disease or disorder associated with a respiratory disease (e.g. symptoms of AERD such as chronic and/or non-allergic rhinitis, nasal polyps, and/or asthma, and the precipitation of both asthma and/or rhinitis attacks after ingestion of aspirin or other COX-1 inhibitors). Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject, e.g. nasal administration, oral administration, administration via aerosol sprays, etc.

The term “statistically significant” or “significantly” refers to statistical significance and generally means a two standard deviation (2SD) difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Immunology by Werner Luttmann, published by Elsevier, 2006. Definitions of common terms in molecular biology can also be found in Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various aspects of the invention.

Described herein are assays and methods of treatment relating to the inventors' discovery that subjects with AERD have elevated levels of platelet-adherent leukocytes as compared to subjects without AERD, e.g. as compared to subjects with aspirin tolerant asthma. As used herein, “aspirin-exacerbated respiratory disease” or “AERD” refers to a condition characterized by the presence of chronic and/or non-allergic rhinitis, nasal polyps, and/or asthma, and the occurrence of both asthma and/or rhinitis attacks after ingestion of aspirin or other COX-1 inhibitors. The aspirin-mediated attacks can be further characterized by involvement of the entire respiratory tract, rhinitis, conjunctivitis, bronchospasm, acute bronchoconstriction, nasal congestion, and/or eye watering (see, e.g. BergesGimene, M. P. et al., Ann Allergy Asthma Immunol 2002; 89:474-478; which is incorporated by reference herein in its entirety). Presently, there is not an effective or safe method to identify a subject with AERD, or any in vitro test to detect aspirin sensitivity in the clinic. Oral challenge with aspirin (which induces an attack) remains the gold standard diagnostic test for AERD. In some embodiments, the subject having AERD or in need of treatment for AERD is a human subject.

As described herein, the inventors have discovered that subjects with AERD can have elevated levels of platelet-adherent leukocytes. As used herein, the term “platelet-adherent leukocyte” refers to a leukocyte which is specifically bound to, or which has the ability to specifically bind to, a platelet. In some embodiments, a platelet-adherent leukocyte can be a platelet-bound leukocyte. In some embodiments, a leukocyte can be specifically bound to a platelet if it binds to the platelet with greater affinity and specificity than it binds to other cell types, e.g. other leukocytes. In some embodiments, a platelet-adherent leukocyte can be identified if the leukocyte remains specifically bound to a platelet during manipulation in standard cell culture reagents (e.g. processing through a FACS machine in a standard eukaryotic cell FACS buffer), where the manipulation does not dissociate the leukocyte and platelet. In some embodiments, a platelet-adherent leukocyte can be a leukocyte that is specifically bound to a platelet where the platelet and leukocyte are bound via a compatible cognate receptor-ligand pair (e.g. P-selectin expressed by platelets and PSGL-1 or other P-selectin ligands expressed by leukocytes; fibrinogen expressed by platelets and β2-integrins expressed by neutrophils; see, e.g. Nash. “Adhesion Between Platelets and Leukocytes or Endothelial Cells” in Platelets and Megakaryocytes: Volume 1: Functional Assays, Series: Methods in Molecular Biology 272: 199-213, Springer: 2004; which is incorporated by reference herein in its entirety).

In some embodiments, a leukocyte is a CD45+ cell, i.e. a cell expressing a detectable level of CD45 on the cell surface. As used herein, “CD45” refers to a cell surface marker comprised by the receptor-type tyrosine-protein phosphatase C polypeptide. The sequence of CD45 for a number of species is well known in the art, e.g. human CD45 (SEQ ID NOs: 1-3; NCBI Ref Seqs: NP 001254727, NP 002829, and NP_{—}563578; NCBI Gene ID: 5788). In some embodiments, a leukocyte is a neutrophil or an eosinophil. In some embodiments, an eosinophil can be a CCR3+ cell, i.e. a cell expressing a detectable level of CCR3 on the cell surface. As used herein, “CCR3” refers to a cell surface marker comprised by the chemokine (C—C motif) receptor 3 polypeptide. The sequence of CCR3 for a number of species is well known in the art, e.g. human CCR3 (SEQ ID NOs: 4-7; NCBI Ref Seqs: NP_{—}001158152, NP_{—}001828, NP_{—}847898, NP_{—}847899; NCBI Gene ID: 1232). In some embodiments, an eosinophil can be a CD45+/CCR3+ cell, i.e. a cell expressing a detectable level of both CD45 and CCR3 on the cell surface. In some embodiments, a neutrophil can be a CD16+ cell, i.e. a cell expressing a detectable level of CD16 on the cell surface. As used herein, “CD16” refers to a cell surface marker comprised by either of two genes; CD16a and CD16b, both of which encode low affinity receptors for the Fc fragment of IgG. The sequences of CD16a and CD16b for a number of species are well known in the art, e.g. human CD16a (SEQ ID NOs: 8-12; NCBI Ref Seqs: NP_{—}000560, NP_{—}001121064, NP_{—}001121065, NP_{—}001121067, NP_{—}001121068; NCBI Gene ID: 2214) and human CD16b (SEQ ID NOs: 13-14; NCBI Ref Seqs: NP_{—}000561, NP_{—}001231682; NCBI Gene ID: 2215). In some embodiments, a neutrophil can be a CD45+/CD16+ cell, i.e. a cell expressing a detectable level of both CD45 and CD16 on the cell surface.

In some embodiments, a leukocyte can be detected using a leukocyte-specific antibody reagent, i.e. an antibody reagent that binds specifically to leukocytes as compared to other cell types comprised by the sample. In some embodiments, a leukocyte-specific antibody reagent can be an anti-CD45 antibody reagent. Anti-CD45 antibody reagents are well known in the art and available commercially, e.g. Cat. No. 10558; AbCam; Cambridge, Mass. In some embodiments, the leukocyte-specific antibody reagent can be an eosinophil-specific reagent, e.g. an antibody reagent that binds to eosinophils and not other cell types comprised by the sample. In some embodiments, the eosinophil-specific reagent can bind specifically to eosinophils, but not to other leukocyte cell types. In some embodiments, an eosinophil-specific reagent can be used in combination with a reagent that binds multiple types of leukocytes (e.g. an anti-CCCR3 antibody reagent and an anti-CD45 antibody reagent can be used concurrently to detect leukocytes generally and/or eosinophils). In some embodiments, the eosinophil-specific reagent can be an anti-CCR3 antibody reagent. Anti-CCR3 antibody reagents are well known in the art and available commercially, e.g. Cat. No. 16231; Abcam; Cambridge, Mass. In some embodiments, the leukocyte-specific antibody reagent can be a neutrophil-specific reagent, e.g. an antibody reagent that binds to neutrophils and not other cell types comprised by the sample. In some embodiments, the neutrophil-specific reagent can bind specifically to neutrophils, but not to other leukocyte cell types. In some embodiments, a neutrophil-specific reagent can be used in combination with a reagent that binds multiple types of leukocytes (e.g. an anti-CD16 antibody reagent and an anti-CD45 antibody reagent can be used concurrently to detect leukocytes generally and/or neutrophils). In some embodiments, the neutrophil-specific reagent can be an anti-CD16 antibody reagent. Anti-CD16 antibody reagents are well known in the art and available commercially, e.g. Cat. No. 664; Abcam; Cambridge, Mass.

In some embodiments, a platelet-adherent leukocyte can be detected using a platelet-specific antibody reagent, e.g. an antibody reagent that binds to platelets and not other cell types comprised by the sample. If a platelet-specific antibody reagent colocalizes to a leukocyte and/or a leukocyte-specific antibody reagent, it can indicate the leukocyte is a platelet-adherent leukocyte. In some embodiments, the platelet-specific antibody reagent can be an anti-CD61 antibody reagent. As used herein, “CD61” refers to a cell surface marker comprised by the integrin beta3 polypeptide. The sequence of CD61 for a number of species is well known in the art, e.g. human CD61 (SEQ ID NO: 15, NCBI Ref Seq: NP-000203; NCBI Gene ID: 3690). Anti-CD61 antibody reagents are well known in the art and available commercially, e.g. Cat. No. 125717; Abcam; Cambridge, Mass. In some embodiments, the platelet-specific reagent can be an anti-CD41 antibody reagent. As used herein, “CD41” refers to a cell surface marker comprised by the integrin alpha 2b polypeptide. The sequence of CD41 for a number of species is well known in the art, e.g. human CD41 (SEQ ID NO:16; NCBI Ref Seq: NP_{—}000410; NCBI Gene ID: 3674). Anti-CD41 antibody reagents are well known in the art and available commercially, e.g. Cat. No. 15021; Abcam; Cambridge, Mass.

In some embodiments, antibody reagents, e.g. antibodies, monoclonal and chimeric antibodies useful in the methods as disclosed herein can be manufactured using well-known methods, e.g., as described in Howard and Kaser “Marking and Using Antibodies: A Practical Handbook” CRC Press (2006); which is incorporated by reference herein in its entirety.

In one aspect, the present technology relates to assays directed to determining if a subject has an increased risk of having AERD. In some embodiments, an assay as described herein can comprise contacting a test sample from a subject with a platelet-specific reagent and measuring the percentage of leukocytes to which the reagent is bound; wherein the binding of the platelet-specific reagent to a leukocyte indicates the presence of a platelet bound to the leukocyte (and thus the presence of a platelet-adherent leukocyte); and wherein an increased level of platelet-adherent leukocytes relative to a reference level indicates the subject has an increased likelihood of having aspirin-exacerbated respiratory disease (AERD).

In some embodiments, the sample can substantially comprise only leukocytes and/or cells bound to leukocytes (e.g. the leukocytes can be isolated from a sample obtained from the subject prior to practicing the methods, assays, and/or systems as described herein, for example, by an immunoaffinity column, flow cytometry sorting by size, or centrifugation). A sample can substantially comprise only leukocytes and/or cells bound to leukocytes if other cells (i.e. any cell which is neither a leukocyte nor bound to a leukocyte) comprise no more than 15% of the cells in the sample, e.g. 15% or less, 10% or less, 5% or less, 2% or less, or 1% or less of the cells in the sample are neither leukocytes nor bound to a leukocyte.

In some embodiments, an assay as described herein can comprise (a) obtaining a test sample comprising leukocytes from peripheral blood of a subject; (b) contacting the sample with a platelet-specific antibody reagent and a leukocyte-specific antibody reagent; and (c) detecting the presence or intensity of a detectable signal associated with individual cells of the sample, e.g., using flow cytometry; wherein the antibody reagents comprise a detectable label or a means of generating a detectable signal; wherein the binding of the platelet-specific antibody reagent to a leukocyte indicates the presence of a platelet-adherent leukocyte; wherein the binding of the platelet-specific antibody reagent to a leukocyte is determined by the colocalization and/or concurrent detection of the platelet-specific antibody reagent and the leukocyte-specific antibody reagent; wherein an increased level platelet-adherent leukocytes within the population of leukocytes, as indicated by the detectable signals, relative to a reference level indicates the subject has an increased risk of having aspirin-exacerbated respiratory disease (AERD).

In some embodiments, described herein is an assay comprising: testing for platelet-adherent leukocyte levels in a sample obtained from a subject or determine if a subject has a likelihood of having asthma-exacerbated respiratory disease (AERD) by using a platelet-specific antibody reagent and a leukocyte-specific antibody reagent; and determining the level of platelet-adherent leukocytes present in the sample; wherein the colocalization of the platelet-specific antibody reagent and the leukocyte-specific antibody reagent indicates the presence of a platelet-adherent leukocyte. In some embodiments, the assay can further comprise: performing the assay to detect the level of platelet-adherent leukocytes in the sample obtained from the subject; measuring the level of platelet-adherent leukocytes in the samples colocalizing with both a platelet-specific antibody reagent and a leukocyte-specific antibody reagent; and identifying a subject having asthma-exacerbated respiratory disease (AERD) based on the level of platelet-adherent leukocytes in the sample, wherein the level of platelet-adherent leukocytes in a sample obtained from a subject with AERD is greater by a statistically significant amount than the level of platelet-adherent leukocytes in a sample obtained from a subject who does not have AERD. In some embodiments, the assay can further comprise: comparing the amount of platelet-adherent leukocytes in the sample obtained from the subject with the level of platelet-adherent leukocytes in a sample obtained from a subject who does not have AERD (e.g. who does not display any signs or symptoms of AERD, or who has aspirin-tolerant asthma) wherein an increase in the level of platelet-adherent leukocytes in the sample obtained from the first subject by at least 2-fold as compared to the subject who does not have AERD is indicative of the subject having, or being at risk of having AERD.

In some embodiments, a platelet-adherent leukocyte can be indicated by the colocalization and/or concurrent detection of a CD61-specific antibody reagent and a CD45-specific reagent. In some embodiments, a platelet-adherent leukocyte can be indicated by the colocalization and/or concurrent detection of a CD61-specific antibody reagent and a CCR3-specific reagent (e.g. a platelet-adherent eosinophil). In some embodiments, a platelet-adherent leukocyte can be indicated by the colocalization and/or concurrent detection of a CD61-specific antibody reagent and a CD16-specific reagent (e.g. a platelet-adherent neutrophil). In some embodiments, a platelet-adherent leukocyte can be indicated by the colocalization and/or concurrent detection of a CD41-specific antibody reagent and a CD45-specific reagent. In some embodiments, a platelet-adherent leukocyte can be indicated by the colocalization and/or concurrent detection of a CD41-specific antibody reagent and a CCR3-specific reagent (e.g. a platelet-adherent eosinophil). In some embodiments, a platelet-adherent leukocyte can be indicated by the colocalization and/or concurrent detection of a CD41-specific antibody reagent and a CD16-specific reagent (e.g. a platelet-adherent neutrophil).

Detection of the presence of platelet-adherent leukocytes and/or determination of the level of platelet-adherent leukocytes as described herein can be according to any method known in the art. Immunological methods to detect platelet-adherent leukocytes in accordance with the present technology include, but are not limited to antibody techniques such as immunohistochemistry, immunocytochemistry, flow cytometry, fluorescent-activated cell sorting (FACS), immunoblotting, radioimmunoassays, western blotting, immunoprecipitation, enzyme-linked immunosorbant assays (ELISA), and derivative techniques that make use of antibody reagents as described herein.

In some embodiments, detection of the presence of platelet-adherent leukocytes and/or determination of the level of platelet-adherent leukocytes can be performed using flow cytometry. In some embodiments, detection of platelet-adherent leukocytes and/or determination of the level of platelet-adherent leukocytes can be performed using immunocytological methods, e.g. FACS.

Flow cytometry is a well-known technique for analyzing and sorting cells (or other small particles) suspended in a fluid stream. This technique allows simultaneous analysis of the physical and/or chemical characteristics of single cells flowing through an optical, electronic, or magnetic detection apparatus. As applied to FACS, the flow cytometer consists of a flow cell which carries the cells in a fluid stream in single file through a light source with excites the fluorescently labeled detection marker(s) (for example, antibody reagents) and measures the fluorescent character of the cell. The fluid stream is then ejected through a nozzle and a charging ring, under pressure, which breaks the fluid into droplets. The flow cell device and fluid stream is calibrated such that there is a relatively large distance between individual cells or bound groups of cells (e.g. a platelet-bound leukocyte), resulting in a low probability that any droplet contains more than a single cell or bound group of cells. The charging ring charges the droplets based on the fluorescence characteristic of the cell which is contained therein. The charged droplets are then deflected by an electrostatically-charged deflection system which diverts the droplets into various containers based upon their charge (related to the fluorescence intensity of the cell). A FACS system (e.g. the FACSARIA™ flow cytometer (BD Biosciences) and FLOWJO™ Version 7.6.4 (TreeStar) as used in the Examples described herein) can detect and record the number of total cells as well as the number of cells which display one or more fluorescent characteristics, e.g. (a) the total number of cells or bound groups of cells in a sample, (b) the number of cells (or bound groups of cells) with a bound leukocyte-specific antibody reagent bound to them, (c) the number of cells (or bound groups of cells) with a platelet-specific antibody reagent bound to them, and (d) the number of cells (or bound groups of cells) which belong to both groups (b) and (c).

In some embodiments, a method, assay, and/or system as described herein can comprise: contacting a sample obtained from a subject with a detectable platelet-specific antibody reagent (comprising a first distinguishable, detectable label) and a detectable leukocyte-specific antibody reagent (comprising a second distinguishable, detectable label), determining whether either and/or both of the distinguishable signals produced by the labels is present on each cell and/or group of cells; wherein the presence of both a signal from a platelet-specific antibody reagent and a signal from a leukocyte-specific reagent on a cell and/or group of cells indicates the presence of a platelet-adherent leukocyte. Stated another way, if the platelet-specific antibody reagent and the leukocyte-specific antibody reagent colocalize or are present or detected on the same complex of cells comprising at least one leukocyte and at least one platelet, it indicates a platelet interacting with the leukocyte and thus the presence of a platelet-adherent leukocyte. In some embodiments, after the contacting step, the sample can be washed to remove the unbound antibody reagents.

An exemplary, non-limiting protocol for determining the level of platelet-adherent leukocytes in a sample using FACS is as follows: whole peripheral blood can be drawn into heparinized tubes, kept at room temperature, and assayed within 1 hour of collection. Ten μL of blood can be incubated with fluorescently conjugated antibodies specific for, e.g., CD61 and a leukocyte-specific marker (e.g. CD45, CD16, or CCR3) or appropriate isotype controls (BD Biosciences) for 20 minutes. The cells can then be fixed in 1% paraformaldehyde. At least 20,000 CD45⁺ cells can be recorded for each sample on an FACSARIA™ flow cytometer (BD Biosciences), and the data analyzed with FLOWJO™ Version 7.6.4 (TreeStar). Within each leukocyte population, the mean fluorescence intensity of each marker can be measured separately for the platelet-adherent subset and the platelet-free subset.

In some embodiments, the level of platelet-adherent leukocytes can be determined using high-throughput FACS (see, e.g. US Patent Publication 2009/0239235 describing a technology commercially available as FACSCANTO™ from BD Biosciences and which is incorporated by reference herein in its entirety).

Immunochemistry is a family of techniques based on the use of a specific antibody, wherein antibodies are used to specifically target molecules inside or on the surface of cells. In some embodiments, immunohistochemistry (“IHC”) and immunocytochemistry (“ICC”) techniques can be used to detect or measure the levels of platelet-adherent leukocytes. IHC is the application of immunochemistry to tissue sections, whereas ICC is the application of immunochemistry to cells or tissue imprints after they have undergone specific cytological preparations such as, for example, liquid-based preparations. In some instances, signal amplification may be integrated into the particular protocol, wherein a secondary antibody, that includes a label, follows the application of an antibody reagent specific for platelets or leukocytes. Typically, for immunohistochemistry, tissue obtained from a subject and fixed by a suitable fixing agent such as alcohol, acetone, and paraformaldehyde, is sectioned and reacted with an antibody. Conventional methods for immunohistochemistry are described in Buchwalow and Bocker (Eds) “Immunohistochemistry: Basics and Methods” Springer (2010): Lin and Prichard “Handbook of Practical Immunohistochemistry” Springer (2011); which are incorporated by reference herein in their entireties. In some embodiments, immunocytochemistry may be utilized where, in general, tissue or cells are obtained from a subject are fixed by a suitable fixing agent such as alcohol, acetone, and paraformaldehyde, to which is reacted an antibody. Methods of immunocytological staining of human samples is known to those of skill in the art and described, for example, in Burry. “Immunocytochemistry: A Practical Guide for Biomedical Research” Springer (2009); which is incorporated by reference herein in its entirety.

Immunochemical methods can include the use of two or more antibodies which will produce a detectable signal only when they are colocalized, e.g. fluorescence resonance energy transfer (FRET) or bioluminescence resonance energy transfer (BRET). Thus, the use of a platelet-specific antibody reagent and leukocyte-specific antibody reagent pair which adapted for use in a FRET protocol will produce a detectable signal only when colocalized on/near a platelet-adherent leukocyte, that is only platelet-adherent leukocytes will generate a detectable signal and only one type of signal must be detected in order to determine the level of platelet-adherent leukocytes present in a sample. FRET and BRET are well known in the art (see, e.g. Daunert and Deo, Eds. “Photoproteins in Bioanalysis” Wiley-VCH: 2006 and Perisamy and Day, Eds. “Molecular Imaging: FRET Microscopy and Spectroscopy” Oxford University Press: 2005: which are incorporated by reference herein in their entireties).

In some embodiments, the assays, methods, and/or systems described herein can comprise: contacting a sample obtained from a subject with a first antibody reagent which is conjugated to a solid support, contacting the sample with a second, detactable antibody reagent, detecting a signal from the second antibody reagent, wherein the presence of a signal from the second antibody reagent indicates the presence of a platelet-adherent leukocyte. In some embodiments, after the contacting steps, the sample can be washed to remove unbound antibody reagents and/or cells not bound to the first antibody reagent and/or groups of cells not bound to the first antibody reagent. In some embodiments, the first antibody reagent can be a platelet-specific antibody reagent and the second antibody reagent can be a leukocyte-specific antibody reagent. In other embodiments, the first antibody reagent can be a leukocyte-specific antibody reagent and the second antibody reagent can be a platelet-specific antibody reagent. In some embodiments, the first antibody reagent can be detectably labeled. In some embodiments, the solid support can comprise a particle (including, but not limited to an agarose or latex bead or particle or a magnetic particle), a bead, a nanoparticle, a polymer, a substrate, a slide, a coverslip, a plate, a dish, a well, a membrane, and/or a grating. The solid support can include many different materials including, but not limited to, polymers, plastics, resins, polysaccharides, silicon or silica based materials, carbon, metals, inorganic glasses, and membranes.

In one embodiment, an assay, method, and/or system as described herein can comprise an ELISA. In an exemplary embodiment, a first antibody reagent can be immobilized on a solid support (usually a polystyrene micro titer plate). The solid support can be contacted with a sample obtained from a subject, and the antibody reagent will bind (“capture”) cells for which it is specific (e.g. either platelets or leukocytes). The solid support can then be contacted with a second labeled antibody reagent (e.g. a detection antibody reagent). The detection antibody reagent can, e.g. comprise a detectable signal, be covalently linked to an enzyme, or can itself be detected by a secondary antibody which is linked to an enzyme through bio-conjugation. The presence of a signal indicates that both the first antibody reagent immobilized on the support and the second “detection” antibody reagent have bound to a cell or group of cells, i.e. the presence of a signal indicates the presence of a platelet-adherent leukocyte. Between each step the plate is typically washed with a mild detergent solution to remove any proteins or antibodies that are not specifically bound. After the final wash step the plate is developed by adding an enzymatic substrate to produce a visible signal, which indicates the quantity of platelet-adherent leukocytes in the sample. Older ELISAs utilize chromogenic substrates, though newer assays employ fluorogenic substrates with much higher sensitivity. In some embodiments, one of the antibody reagents can be a platelet-specific antibody reagent and one of the antibody reagents can be a leukocyte-specific antibody reagent. There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. 1984, J. Clin. Chem. Clin. Biochem. 22:895-904. These references are hereby incorporated by reference in their entirety.

In some embodiments, described herein is an assay to detect the amount of colocalization of a platelet and a leukocyte in a subject, the assay comprising: (a) contacting a sample obtained from the subject with a platelet-specific antibody reagent and at least one leukocyte-specific antibody reagent; (b) measuring the amount of colocalization of the signal from the platelet-specific antibody reagent with the signal from the leukocyte-specific antibody reagent, wherein colocalization identifies the presence of a platelet-adherent leukocyte; (c) optionally, comparing the amount of platelet-adherent leukocytes with a reference level, and wherein if the level of platelet-adherent leukocytes is increased (e.g. increased by at least 2-fold compared to the reference level) the subject is identified as having, at risk of having, or being in need of treatment for AERD.

In one embodiment, the assays, systems, and methods described herein can comprise a lateral flow immunoassay test (LFIA), also known as the immunochromatographic assay, or strip test to measure or determine the level of platelet-adherent leukocytes in a sample. LFIAs are a simple device intended to detect the presence (or absence) of platelet-adherent leukocytes in a sample. There are currently many LFIA tests are used for medical diagnostics either for home testing, point of care testing, or laboratory use. LFIA tests are a form of immunoassay in which the test sample flows along a solid substrate via capillary action. After the sample is applied to the test it encounters a colored antibody reagent which mixes with the sample, and if bound to a portion of the sample, transits the substrate encountering lines or zones which have been pretreated with a second antibody reagent. Depending upon the level of platelet-adherent leukocytes present in the sample the colored antibody reagent can become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water samples etc. Strip tests are also known as dip stick test, the name bearing from the literal action of “dipping” the test strip into a fluid sample to be tested. LFIA strip test are easy to use, require minimum training and can easily be included as components of point-of-care test (POCT) diagnostics to be use on site in the field. LFIA tests can be operated as either competitive or sandwich assays. Sandwich LFIAs are similar to sandwich ELISA. The sample first encounters colored particles which are labeled with antibody reagents specific for a target (e.g. a platelet-specific antibody reagent or a leukocyte-specific antibody reagent). The test line will also contain antibody reagents (e.g. a platelet-specific antibody reagent or a leukocyte-specific antibody reagent). The test line will show as a colored band in positive samples. In some embodiments, the lateral flow immunoassay can be a double antibody sandwich assay, a competitive assay, a quantitative assay or variations thereof. There are a number of variations on lateral flow technology. It is also possible to apply multiple capture zones to create a multiplex test.

A typical test strip consists of the following components: (1) sample application area comprising an absorbent pad (i.e. the matrix or material) onto which the test sample is applied; (2) conjugate or reagent pad—this contains antibody reagent(s) specific to the target which can be conjugated to colored particles (usually colloidal gold particles, or latex microspheres); (3) test results area comprising a reaction membrane—typically a hydrophobic nitrocellulose or cellulose acetate membrane onto which antibody reagents are immobilized in a line across the membrane as a capture zone or test line (a control zone may also be present, containing antibodies specific for the antibody reagents conjugated to the particles or microspheres); and (4) optional wick or waste reservoir—a further absorbent pad designed to draw the sample across the reaction membrane by capillary action and collect it. The components of the strip are usually fixed to an inert backing material and may be presented in a simple dipstick format or within a plastic casing with a sample port and reaction window showing the capture and control zones. While not strictly necessary, most tests will incorporate a second line which contains an antibody that picks up free latex/gold in order to confirm the test has operated correctly.

The use of “dip sticks” or LFIA test strips and other solid supports have been described in the art in the context of an immunoassay for a number of antigen biomarkers. U.S. Pat. Nos. 4,943,522; 6,485,982; 6,187,598; 5,770,460; 5,622,871; 6,565,808, U.S. patent application Ser. No. 10/278,676; U.S. Ser. No. 09/579,673 and U.S. Ser. No. 10/717,082, which are incorporated herein by reference in their entirety, are non-limiting examples of such lateral flow test devices. Three U.S. patents (U.S. Pat. No. 4,444,880, issued to H. Tom; U.S. Pat. No. 4,305,924, issued to R. N. Piasio; and U.S. Pat. No. 4,135,884, issued to J. T. Shen) describe the use of “dip stick” technology to detect soluble antigens via immunochemical assays. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teaching of these “dip stick” technology for the detection of platelet-adherent leukocytes.

Further examples of protocols which can be used in the methods, assays, and systems described herein to detect platelet-adherent leukocytes are described in U.S. Pat. No. 6,586,259; Li et al. Cytometry. 1999 35:154-161; and Hagberg and Lyberg. Platelets. 2000 11:151-160; which are incorporated herein by reference in their entireties. Unlike the present invention, U.S. Pat. No. 6,586,259, Li et al. and Hagberg and Lyberg do not discuss measuring the level of platelet-adherent leukocytes to identify or treat a subject having AERD.

In some embodiments, one or more of the antibody reagents described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into an antibody reagent are well known in the art.

In some embodiments, detectable labels can include labels that can be detected by spectroscopic, photochemical, biochemical, immunochemical, electromagnet, radiochemical, or chemical means, such as fluorescence, chemifluorescence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the antibody reagent. Alternatively, a detectable label can be linked such as by directly labeling a molecule that achieves binding to the antibody reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection antibody is label with a fluorescent compound. When the fluorescently labeled antibody is exposed to light of the proper wavelength, its presence can then be detected due to fluorescence. In some embodiments, a detectable label can be a fluorescent dye molecule, qtr fluorophore including, but not limited to fluorescein, phycoerythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyflorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g. umbelliferone benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes.

In some embodiments, a detectable label can be a radiolabel including, but not limited to ³H, ¹²⁵I, ³⁵S, ¹⁴C, ³²P, and ³³P.

In some embodiments, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

In some embodiments, a detectable label is a chemiluminescent label, including, but not limited to lucigenin luminol, luciferin, isoluminol, theromatic acridinium ester, imidazole, acridinium salt and oxalate ester.

In some embodiments, a detectable label can be a spectral colorimetric label including, but not limited to colloidal gold or colored glass or plastic (e.g., polystyrene, polypropylene, and latex) beads.

In some embodiments, antibodies can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i.e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e.g. from DAKO; Carpinteria, Calif.

An antibody reagent can also be detectably labeled using fluorescence emitting metals such as ¹⁵²Eu, or others of the lanthanide series. These metals can be attached to the antibody reagent using such metal chelating groups as diethylenetriaminepentaacetic acid (DTPA) or ethylenediaminetetraacetic acid (EDTA).

In some embodiments, wherein at least two different antibody reagents are used (e.g. a platelet-specific reagent and a leukocyte-specific reagent), the different types of antibody reagents can be labeled with different detectable labels. Two detectable labels are considered different if the signal from one label can be distinguished from the signal from the other.

The assays and methods as described herein can relate to determining if a subject has an increased level of platelet-adherent leukocytes relative to a reference level. In some embodiments, the reference level can comprise the level of platelet-adherent leukocytes in a sample of the same type taken from a subject not exhibiting any signs or symptoms of a respiratory disease, e.g. asthma. In some embodiments, the reference level of platelet-adherent leukocytes can be the level of platelet-adherent leukocytes in a healthy subject not having, or not diagnosed as having, a respiratory disease. In some embodiments, the reference level of platelet-adherent leukocytes can be the level of platelet-adherent leukocytes in a subject having, or diagnosed as having, aspirin-tolerant asthma. In some embodiments, the reference level can be the level in a sample of similar cell type, sample type, sample processing, and/or obtained from a subject of similar age, sex and other demographic parameters as the sample/subject for which the level of platelet-adherent leukocytes is to be determined. In some embodiments, the test sample and control reference sample are of the same type, that is, obtained from the same biological source, and comprising the same composition, e.g. the same number and type of cells.

In some embodiments, a level of platelet-adherent leukocytes can be increased relative to a reference level if the level of platelet-adherent leukocytes is at least 2× of the reference level, e.g. at least 2×, at least 3×, at least 4×, at least 5×, at least 6×, or greater of the reference level. In some embodiments, a level of platelet-adherent leukocytes can be increased relative to a reference level if the level of platelet-adherent leukocytes is at least 3× of the reference level. In some embodiments, a level of platelet-adherent leukocytes can be increased relative to a reference level if at least 15% of the leukocytes in the sample are platelet-adherent leukocytes, e.g. at least 15%, at least 20%, at least 25%, at least 30% or more of the leukocytes in the sample are platelet-adherent leukocytes. In some embodiments, a level of platelet-adherent leukocytes can be increased relative to a reference level if at least 50% of the eosinophils in the sample are platelet-adherent eosinophils, e.g. at least 50%, at least 55%, at least 60%, at least 65% or more of the eosinophils in the sample are platelet-adherent eosinophils. In some embodiments, a level of platelet-adherent leukocytes can be increased relative to a reference level if at least 25% of the neutrophils in the sample are platelet-adherent neutrophils, e.g. at least 25%, at least 30%, at least 35%, at least 40% or more of the neutrophils in the sample are platelet-adherent neutrophils.

In some embodiments, a level of platelet-adherent leukocytes in a subject identified to have AERD is increased relative to a reference level by at least 2× of the reference level, e.g. at least 2×, at least 3×, at least 4×, at least 5×, at least 6×, or greater of the reference level. In some embodiments, a level of platelet-adherent leukocytes in a subject identified to have AERD is increased relative to a reference level by at least 3× of the reference level. In some embodiments, a level of platelet-adherent leukocytes in a subject identified to have AERD is increased relative to a reference level if at least 15% of the leukocytes in the sample are platelet-adherent leukocytes, e.g. at least 15%, at least 20%, at least 25%, at least 30% or more of the leukocytes in the sample are platelet-adherent leukocytes. In some embodiments, a level of platelet-adherent leukocytes in subject identified to have AERD is increased relative to a reference level if at least 50% of the eosinophils in the sample are platelet-adherent eosinophils, e.g. at least 50%, at least 55%, at least 60%, at least 65% or more of the eosinophils in the sample are platelet-adherent eosinophils. In some embodiments, a level of platelet-adherent leukocytes in a subject identified to have AERD is increased relative to a reference level if at least 25% of the neutrophils in the sample are platelet-adherent neutrophils, e.g. at least 25%, at least 30%, at least 35%, at least 40% or more of the neutrophils in the sample are platelet-adherent neutrophils.

The term “sample” or “test sample” as used herein denotes a sample taken or isolated from a biological organism, e.g., a blood sample from a subject. Exemplary biological samples include, but are not limited to, whole blood; peripheral blood; whole peripheral blood; a nasal polyp; etc. The term also includes a mixture of the above-mentioned samples. The term “test sample” also includes untreated or pretreated (or pre-processed) biological samples. In some embodiments, a test sample can comprise cells from subject. In some embodiments, a test sample can comprise leukocytes.

In some embodiments, the sample can comprise a biological tissue selected from the group consisting of: whole blood; peripheral blood; whole peripheral blood; a nasal polyp; and products thereof. In some embodiments, the sample can comprise any tissue affected by, or suffering from symptoms, or display markers of AERD, e.g. the sample can comprise bronchial biopsies and/or gastrointestinal samples.

The test sample can be obtained by removing a sample of cells from a subject, but can also be accomplished by using previously isolated cells (e.g. isolated at a prior timepoint and isolated by the same or another person). In addition, the test sample can be freshly collected or a previously collected sample.

In some embodiments, the test sample can be an untreated test sample. As used herein, the phrase “untreated test sample” refers to a test sample that has not had any prior sample pretreatment except for dilution and/or suspension in a solution. Exemplary methods for treating a test sample include, but are not limited to, centrifugation, filtration, sonication, homogenization, heating, freezing and thawing, and combinations thereof. In some embodiments, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments, the test sample is a clarified test sample, for example, by centrifugation and collection of a supernatant comprising the clarified test sample. In some embodiments, a test sample can be a pre-processed test sample, for example, supernatant or filtrate resulting from a treatment selected from the group consisting of centrifugation, filtration, thawing, purification, and any combinations thereof. In some embodiments, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of platelet-adherent leukocytes as described herein.

In some embodiments, the methods, assays, and systems described herein can further comprise a step of obtaining a test sample from a subject.

In some embodiments, the methods, assays, and systems described herein can comprise creating a report based on the level of platelet-adherent leukocytes. In some embodiments, the report denotes raw values of the number/level of platelet-adherent leukocytes in the test sample (plus, optionally, the number/level of platelet-adherent leukocytes in a reference sample) or it indicates a percentage or fold increase in platelet-adherent leukocytes as compared to a reference level, and/or provides a signal that the subject is at risk of having, or not having AERD.

The methods, assays, and systems described herein can relate to methods of treatment, methods of determining if a subject can benefit from certain therapies, and/or methods of determining if a subject should not be given, and/or should avoid or be counseled to avoid certain therapies (e.g. COX-1 inhibitors).

As subjects with AERD will experience a respiratory crisis and/or exacerbation of respiratory disease symptoms following administration of a cyclooxygenase (COX-1) inhibitor, e.g. aspirin, subjects having AERD or at increased risk of having or developing AERD should not be administered a COX-1 inhibitor. Non-limiting examples of COX-1 inhibitors can include aspirin; diclofenac; ibuprofen; naproxen; mefenamic acid; indomethacin; ketoprofen; piroxicam; diflunisal; salsalate; dexibuprofen; fenoprofen; dexketoprofen; flurbiprofen; oxaprozin; loxoprofen; indomethacin; sulindac; etodolac; ketorolac; nabumetone; meloxicam; tenoxicam; droxicam; lornoxicam; isoxicam; mefenamic acid; meclofenamic acid; flufenamic acid; and tolfenamic acid. As used herein, “administration of a COX-1 inhibitor” refers to administration of normal doses of COX-1 inhibitors and specifically excludes aspririn desensitization and high-dose aspirin therapy as described below herein.

Conversely, treatments for AERD, including those known to one of ordinary skill in the art, can be administered to a subject identified as having AERD or at increased risk of having or developing AERD. Non-limiting examples of therapies for AERD can include aspirin desensitization and high-dose aspirin therapy (see, e.g. Rozsasi et al. Allergy 2008 63:1228-34; Lee et al. J Allergy and Clin Immunol 2007 119:157-164; Williams and Woessner. Curr Allergy Astham Rep 2008 8:245-52; and Baker and Quinn. Allergy Asthma Proc 2011 32:335-40; each of which is incorporated by reference herein in its entirety); a P2Y12 inhibitor; a leukotriene receptor antagonist; montelukast; a thromboxane receptor antagonist; a 5-lipoxygenase inhibitor; and zileuton. Non-limiting examples of P2Y12 inhibitors can include clopidogrel (PLAVIX™), cangrelor, ticagrelor, ticlopidine (TICLID™), prasugrel (EFFIENT™), elinogrel (PRT060128 or PRT128. P2Y12 inhibitors and methods of making them are described, for example, in the following patent publications; WO/2006/073361, WO/2008/062770, WO/2008/004944, WO/2007/105751, WO/2006/077851, US2008/0108635, US2009/0048216, PCT/US06/43093, EP Patent Nos. 2,138482, and U.S. Pat. No. 7,488,739; which are incorporated herein by reference in their entireties. Non-limiting examples of 5-lipoxygenase inhibitors can include azelastine (ASTELIN™, ASTELPRO™), diethylcarbamazine, nordihydroguaiaretic acid, and zileuton (ZYFLO™). Non-limiting examples of leukotriene receptor antagonists can include montelukast (SINGULAIR™) and zafirlukast (ACCOLATE™). Thromboxane receptor antagonists can include, but are not limited to, iretroban (HEPATOREN™), AA-2414 (SERATRODAST™), S18886 (terutroban), PTA2, 13-APA, GR-32191, BM-13177 (sulotroban), SQ-29,548, SQ-28,668, ONO-3708, Bay U3405, EP-045, BMS-180,291, S-145, I-BOP ([1S-[1alpha,2alpha(Z),3beta(1E,3S*),4alpha]]-7-[3-[3-hydroxy-4-(4-iodophenoxy)-1-butenyl]-7-oxabi-cyclo[2.2.1]hept-2-yl]5-heptenoic acid), U46619 (9,11-dideoxy-9alpha11alpha-methanoepoxy-prosta-5Z,13E-dien-1-oic acid), PBT-3 [10(S)-hydroxy-11,12-cyclopropyl-eicosa-5Z,8Z,14Z-trienoic acid methyl ester], hepoxilin cyclopropane, BM-531 (N-tert-butyl-N′-[(2-cyclohexylamino-5-nitrobenzene)sulfonyl]urea), EV-077, L0655, and ICI 192,605. Thromboxane receptor antagonists and methods of making them are described, e.g. in International Patent Publication No. WO 2012/009545; U.S. Patent Publication No. 2009/0012115; and U.S. Pat. Nos. 4,443,477; 4,752,616; 4,839,384; 5,066,480; 5,100,889; 5,312,818; 5,399,725; and 6,509,348.

In some aspects, the methods and assays described herein can relate to administering a treatment to a subject with a respiratory disease, administering a treatment to a subject with AERD, determining if a subject will benefit from treatment with AERD therapies, determining if a subject is at increased risk of having AERD, and/or determining if a subject with a respiratory disorder should not be administered a COX-1 inhibitor. These assay, methods, and systems as described herein relate to determining the level of platelet-adherent leukocytes in a sample obtained from the subject. In some embodiments, the methods do not comprise challenging the subject with aspirin or another COX-1 inhibitor.

In some embodiments, the present technology relates to a method of administering a treatment to a subject with a respiratory disease, the method comprising contacting a test sample from the subject with a platelet-specific antibody reagent; measuring the percentage of leukocytes to which the reagent is bound; wherein the binding of the reagent to a leukocyte indicates that the leukocyte is a platelet-bound leukocyte; and administering a therapy for aspirin-exacerbated respiratory disease (AERD) if the level of platelet-bound leukocytes is increased (e.g. at least 2-fold) relative to a reference level; wherein the therapy for AERD is selected from the group consisting of aspirin desensitization and high-dose aspirin therapy; a P2Y12 inhibitor; montelukast; a thromboxane receptor antagonist; a 5-lipoxygenase inhibitor; and zileuton; and wherein the subject is not administered aspirin or a COX-1 inhibitor. In some embodiments, the method can comprise contacting a test sample from the subject with a platelet-specific antibody reagent and a leukocyte-specific antibody reagent; and measuring the percentage of leukocytes to which the reagent is bound; wherein the binding of the platelet-specific antibody reagent to a leukocyte indicates the presence of a platelet-adherent leukocyte; wherein the binding of the platelet-specific antibody reagent to a leukocyte is determined by the colocalization and/or concurrent detection of the platelet-specific antibody reagent and the leukocyte-specific antibody reagent; and administering a therapy for aspirin-exacerbated respiratory disease (AERD) if the level of platelet-bound leukocytes is increased relative to a reference level; wherein the therapy for AERD is selected from the group consisting of aspirin desensitization and high-dose aspirin therapy; a P2Y12 inhibitor; montelukast; a thromboxane receptor antagonist; a 5-lipoxygenase inhibitor; and zileuton; and wherein the subject is not administered aspirin or a COX-1 inhibitor.

In some embodiments, the present technology relates to a method of identifying a subject with a respiratory disease who will benefit from treatment a therapy for aspirin-exacerbated respiratory disease (AERD), the method comprising: contacting a test sample from the subject with a platelet-specific antibody reagent; and measuring the percentage of leukocytes to which the reagent is bound; wherein the binding of the reagent to a leukocyte indicates that the leukocyte is a platelet-bound leukocyte; wherein the subject is identified as needing treatment with a therapy for AERD if the level of platelet-bound leukocytes is increased (e.g. at least 2-fold) relative to a reference level; wherein the therapy for AERD is selected from the group consisting of: aspirin desensitization and high-dose aspirin therapy; a P2Y12 inhibitor; montelukast; a thromboxane receptor antagonist; a 5-lipoxygenase inhibitor; and zileuton and wherein the subject is not administered aspirin or a COX-1 inhibitor. In some embodiments, the method can comprise contacting a test sample from the subject with a platelet-specific antibody reagent and a leukocyte-specific antibody reagent; and measuring the percentage of leukocytes to which the reagent is bound; wherein the binding of the platelet-specific antibody reagent to a leukocyte indicates the presence of a platelet-adherent leukocyte; wherein the binding of the platelet-specific antibody reagent to a leukocyte is determined by the colocalization and/or concurrent detection of the platelet-specific antibody reagent and the leukocyte-specific antibody reagent; wherein the subject is identified as needing treatment with a therapy for AERD if the level of platelet-bound leukocytes is increased relative to a reference level; wherein the therapy for AERD is selected from the group consisting of: aspirin desensitization and high-dose aspirin therapy; a P2Y12 inhibitor; montelukast; a thromboxane receptor antagonist; a 5-lipoxygenase inhibitor; and zileuton; and wherein the subject is not administered aspirin or a COX-1 inhibitor.

In some embodiments, the present technology relates to a method of determining if a subject is at increased risk of having aspirin-exacerbated respiratory disease (AERD), the method comprising: measuring the percentage of platelet-bound leukocytes in a sample obtained from the subject; wherein an increased level of platelet-bound leukocytes relative to a reference level indicates the subject is at increased risk of having AERD. In some embodiments, the method comprises contacting a sample obtained from the subject with a platelet-specific antibody reagent and a leukocyte-specific antibody reagent and detecting the presence or intensity of a detectable signal associated with individual cells of the sample, e.g., using flow cytometry; wherein the antibody reagents comprise a detectable label or a means of generating a detectable signal; wherein the binding of the platelet-specific antibody reagent to a leukocyte indicates the presence of a platelet-adherent leukocyte; wherein the binding of the platelet-specific antibody reagent to a leukocyte is determined by the colocalization and/or concurrent detection of the platelet-specific antibody reagent and the leukocyte-specific antibody reagent; wherein an increased level platelet-adherent leukocytes within the population of leukocytes, as indicated by the detectable signals, relative to a reference level indicates the subject has an increased risk of having aspirin-exacerbated respiratory disease (AERD). In some embodiments a subject identified as being at increased risk of having AERD can be administered a therapy for AERD. In some embodiments, a subject identified as being at increased risk of having AERD can be instructed to avoid and/or not be administered a COX-1 inhibitor.

In some embodiments, the present technology relates to an assay to determine if a subject with a respiratory disease should not be administered a cyclooxygenase-1 (COX1) inhibitor, the assay comprising: contacting a test sample from the subject with a platelet-specific reagent and measuring the percentage of leukocytes to which the reagent is bound; wherein the binding of the platelet-specific reagent to a leukocyte indicates the presence of a platelet bound to the leukocyte; and wherein an increased level of platelet-adherent leukocytes relative to a reference level indicates the subject should not be administered a COX1 inhibitor. In some embodiments, the present technology relates to an assay to determine if a subject with a respiratory disease will benefit from treatment with an aspirin-exacerbated respiratory disease (AERD) therapy, the assay comprising: contacting a test sample from the subject with a platelet-specific reagent and measuring the percentage of leukocytes to which the reagent is bound; wherein the binding of the platelet-specific reagent to a leukocyte indicates the presence of a platelet bound to the leukocyte; and wherein an increased level of platelet-adherent leukocytes relative to a reference level indicates the subject will benefit from treatment with an AERD therapy. In some embodiments, the method comprises contacting a sample obtained from the subject with a platelet-specific antibody reagent and a leukocyte-specific antibody reagent and detecting the presence or intensity of a detectable signal associated with individual cells of the sample, e.g., using flow cytometry; wherein the antibody reagents comprise a detectable label or a means of generating a detectable signal; wherein the binding of the platelet-specific antibody reagent to a leukocyte indicates the presence of a platelet-adherent leukocyte; wherein the binding of the platelet-specific antibody reagent to a leukocyte is determined by the colocalization and/or concurrent detection of the platelet-specific antibody reagent and the leukocyte-specific antibody reagent; wherein an increased level platelet-adherent leukocytes within the population of leukocytes, as indicated by the detectable signals, relative to a reference level indicates the will benefit from treatment with an AERD therapy.

In some embodiments, described herein is a method of treating AERD, the method comprising administering a treatment selected from the group consisting of a P2Y12 inhibitor; a leukotriene receptor antagonist; montelukast; a thromboxane receptor antagonist; a 5-lipoxygenase inhibitor; and zileuton to a subject in need of treatment for AERD. In some embodiments, the subject in need of treatment for AERD is a subject identified to have AERD as described herein, e.g. a subject with an increased number of platelet-adherent leukocytes present in their blood.

As demonstrated herein, the level of platelet-adherent leukocytes contributes to the pathophysiology of AERD, e.g. by secreting higher levels of leukotrienes. Accordingly, described herein is a method of treating AERD by administering a treatment that reduces the level of platelet-adherent leukocytes, e.g. a treatment that inhibits the binding of platelets to leukocytes and/or the binding of leukocytes to platelets. In some embodiments, the treatment can be selected from the group consisting of a P2Y12 inhibitor; a leukotriene receptor antagonist; montelukast; a thromboxane receptor antagonist; a 5-lipoxygenase inhibitor; and zileuton.

In one aspect, described herein is a method of monitoring and/or directing the treatment of a subject in need of treatment for AERD, the method comprising: a) measuring a first level of platelet-bound leukocytes in a sample obtained from a subject as described herein; b) administering a treatment for AERD if the subject is determined to have an increased level of platelet-bound leukocytes relative to a reference; c) measuring a second level of platelet-bound leukocytes in a sample obtained from a subject as described herein; and adjusting the dosage of the treatment as indicated by the second level of platelet-bound leukocytes. In some embodiments, steps b) and c) can be repeated multiple times, e.g. twice, three times, four times, or more times. In some embodiments, steps b) and c) can be repeated on a regular basis, e.g. every day, every week, every two weeks, once a month, once every two months or less often. In some embodiments, if the second level of platelet-bound leukocytes is the same or greater than the first level, the subject can be administered a greater dose and/or increased frequency of treatment. In some embodiments, if the second level of platelet-bound leukocytes is less than the first level, the subject can be administered a smaller dose and/or decreased frequency of treatment. In some embodiments, if the second level of platelet-bound leukocytes is less than the first level but still greater than the reference level, the subject can be administered a smaller dose and/or decreased frequency of treatment. In some embodiments, if the second level of platelet-bound leukocytes is less than the first level and not statistically significantly higher than the reference level, the subject can be administered a smaller dose and/or decreased frequency of treatment. In some embodiments, if the second level of platelet-bound leukocytes is less than the first level and not statistically significantly higher than the reference level, the subject is not administered any further treatment for AERD. In some embodiments, if the second level of platelet-bound leukocytes is less than the first level and not statistically significantly higher than the reference level, the subject can be administered a different treatment. In some embodiments, if the second level of platelet-bound leukocytes is greater than or equal to the first level, the subject can be administered a different treatment. In some embodiments, the treatment can be selected from the group consisting of a P2Y12 inhibitor; a leukotriene receptor antagonist; montelukast; a thromboxane receptor antagonist; a 5-lipoxygenase inhibitor; and zileuton.

In some embodiments, the methods described herein relate to treating a subject having or diagnosed as having a respiratory disease with an AERD therapy. Subjects having respiratory disease can be identified by a physician using current methods of diagnosing respiratory disease, e.g. asthma. Symptoms and/or complications of asthma which characterize these conditions and aid in diagnosis are well known in the art and include but are not limited to, wheezing, coughing, difficulty breathing, tightness in the chest, and nocturnal worsening of symptoms. Tests that may aid in a diagnosis of, e.g. asthma include, but are not limited to, pulmonary function tests, exhaled nitric oxide test, or tests to rule out other conditions (e.g. x-rays and/or CT scans to rule out COPD or congestive heart failure). A family history of asthma can also aid in determining if a subject is likely to have asthma (or AERD) or in making a diagnosis.

The compositions and methods described herein can be administered to a subject having or diagnosed as having a respiratory disease, asthma, and/or AERD. In some embodiments, the methods described herein comprise administering an effective amount of compositions described herein, e.g. an AERD therapy to a subject in order to alleviate a symptom of AERD. As used herein, “alleviating a symptom of AERD” is ameliorating any condition or symptom associated with AERD. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral and airway (aerosol), administration. Administration can be local or systemic.

The term “effective amount” as used herein refers to the amount of AERD therapy needed to alleviate at least one or more symptom of the disease or disorder, and relates to a sufficient amount of pharmacological composition to provide the desired effect. The term “therapeutically effective amount” therefore refers to an amount of an AERD therapy that is sufficient to effect a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of an AERD therapeutic which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, an effective dose of a composition comprising an AERD therapy as described herein can be administered to a patient once. In certain embodiments, an effective dose of a composition comprising an AERD therapy can be administered to a patient repeatedly. For systemic administration, subjects can be administered a therapeutic amount of a composition comprising an AERD therapy such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more. A composition comprising an AERD therapy can be administered over a period of time, such as over a 5 minute, 10 minute, 15 minute, 20 minute, or 25 minute period. The administration can be repeated, for example, on a regular basis, such as hourly for 3 hours, 6 hours, 12 hours or longer or such as biweekly (i.e., every two weeks) for one month, two months, three months, four months or longer.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. AERD by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the therapeutic. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more.

In some aspects, the invention described herein is directed to systems (and computer readable media for causing computer systems) for obtaining data from at least one sample obtained from at least one subject, the system comprising 1) a measuring module configured to measure the level of platelet-bound leukocytes in a population of leukocytes in a test sample obtained from a subject, 2) a storage module configured to store output data from the measuring module, 3) a comparison module adapted to compare the data stored on the storage module with a reference level, and to provide a retrieved content, and 4) a display module for displaying whether the level of platelet-bound leukocytes in a population of leukocytes in a test sample obtained from a subject is greater, by a statistically significant amount, than the reference level and/or displaying the relative levels of platelet-bound leukocytes.

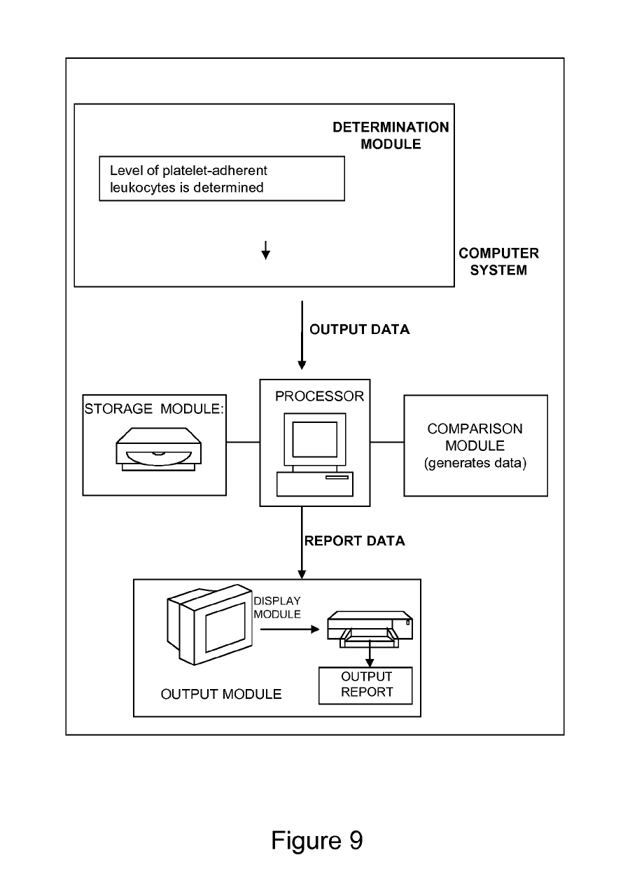

In one embodiment, provided herein is a system comprising: (a) at least one memory containing at least one computer program adapted to control the operation of the computer system to implement a method that includes 1) a measuring module configured to measure the level of platelet-bound leukocytes in a population of leukocytes in a test sample obtained from a subject, 2) a storage module configured to store output data from the measuring module, 3) a computing module adapted to identify from the output data whether the level of platelet-adherent leukocytes in a sample obtained from a subject is statistically significantly greater than a reference level, and 4) a display module for displaying a content based in part on the data output from the measuring module, wherein the content comprises a signal indicative of the level of platelet-adherent leukocytes and (b) at least one processor for executing the computer program (see FIG. 9).

In some embodiments, the measuring module can measure the presence and/or intensity of a detectable signal from an immunoassay indicating the presence of platelet-specific antibody reagent on the leukocytes in the test sample. In some embodiments, the measuring module can measure the presence and/or intensity of a detectable signal from an immunoassay indicating the presence of leukocyte-specific antibody reagent on the cells in the test sample. Exemplary embodiments of a measuring module can include a FACS machine, automated immunoassay, etc.

The measuring module can comprise any system for detecting a signal elicited from an assay to determine the level of platelet-adherent leukocytes as described above herein. In some embodiments, such systems can include an instrument, e.g., FACSARIA™ (BD Biosciences) as described herein for FACS analysis. In another embodiment, the measuring module can comprise multiple units for different functions, such as measurement of platelets (and/or detectable signals from platelet-specific antibody reagents) and measurement of leukocytes (and/or detectable signals from leukocyte-specific antibody reagents). In one embodiment, the measuring module can be configured to perform the methods described elsewhere herein, e.g. FACS, or detection of any detectable label or signal.

In some embodiments, the measuring system or a further module can be configured to process whole blood samples, e.g. to separate cells or portions of cells from whole blood for use in the assays described herein.

The term “computer” can refer to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” may refer to any storage device used for storing data accessible by a computer, as well as any other means for providing access to data by a computer. Examples of a storage-device-type computer-readable medium include: a magnetic hard disk; a floppy disk; an optical disk, such as a CD-ROM and a DVD; a magnetic tape; a memory chip. The term a “computer system” may refer to a system having a computer, where the computer comprises a computer-readable medium embodying software to operate the computer. The term “software” is used interchangeably herein with “program” and refers to prescribed rules to operate a computer. Examples of software include: software; code segments; instructions; computer programs; and programmed logic.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

Embodiments of the invention can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules can perform other functions, thus the modules are not limited to having any particular functions or set of functions.

The functional modules of certain embodiments of the invention include at minimum a measuring module, a storage module, a computing module, and a display module. The functional modules can be executed on one, or multiple, computers, or by using one, or multiple, computer networks. The measuring module has computer executable instructions to provide e.g., levels of platelet-adherent leukocytes etc. in computer readable form.

The information determined in the measuring system can be read by the storage module. As used herein the “storage module” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon, for example, sample name, biomolecule assayed and the level of said biomolecule. Such information may be provided in digital form that can be transmitted and read electronically, e.g., via the Internet, on diskette, via USB (universal serial bus) or via any other suitable mode of communication.

As used herein, “stored” refers to a process for encoding information on the storage module. Those skilled in the art can readily adopt any of the presently known methods for recording information on known media to generate manufactures comprising expression level information.

In some embodiments of any of the systems described herein, the storage module stores the output data from the measuring module. In additional embodiments, the storage module stores reference information such as levels of platelet-adherent leukocytes in healthy subjects, subjects not having a respiratory disorder and/or a population of subjects with aspirin tolerant asthma.

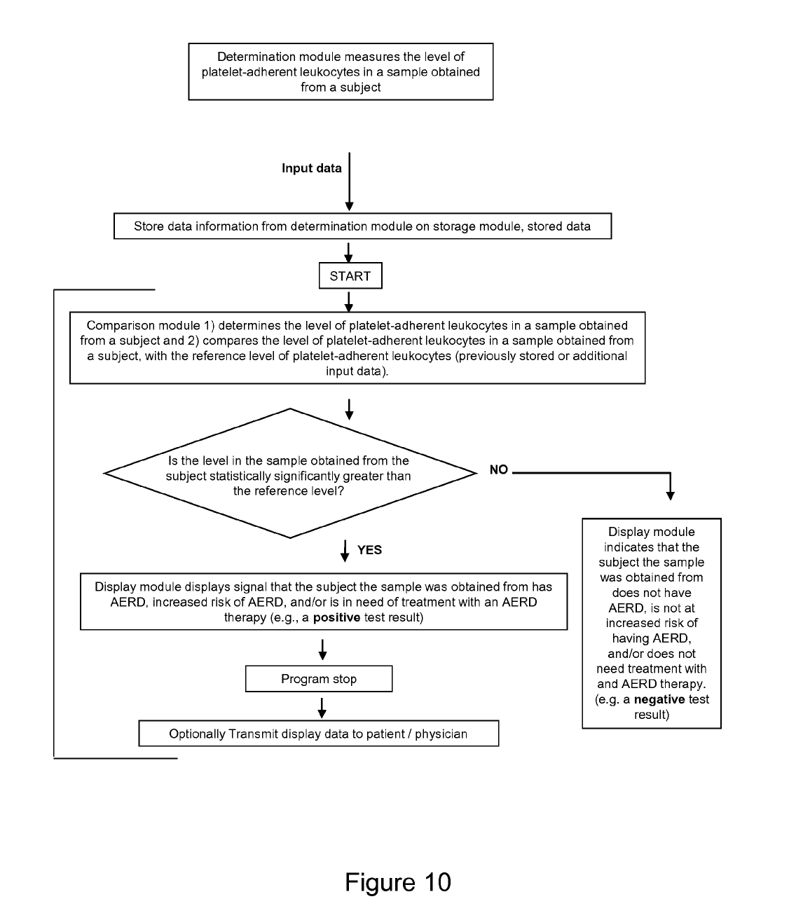

The “computing module” can use a variety of available software programs and formats for computing the level of platelet-adherent leukocytes. Such algorithms are well established in the art. A skilled artisan is readily able to determine the appropriate algorithms based on the size and quality of the sample and type of data. The data analysis tools and equations described herein can be implemented in the computing module of the invention. In some embodiments, the computing module can comprise a computer and/or a computer system. In one embodiment, the computing module further comprises a comparison module, which compares the level of platelet-adherent leukocytes in a sample obtained from a subject as described herein with a reference level as described herein (see, e.g. FIG. 10). By way of an example, when the level of platelet-adherent leukocytes in a sample obtained from a subject is measured, a comparison module can compare or match the output data with the mean level of platelet-adherent leukocytes in a population of subjects not having signs or symptoms of a respiratory disorder (i.e. a reference level). In certain embodiments, the mean level of platelet-adherent leukocytes in a population of subjects not having signs or symptoms of a respiratory disorder can be pre-stored in the storage module. During the comparison or matching process, the comparison module can determine whether the level of platelet-adherent leukocytes in a sample obtained from a subject is statistically significantly greater than the reference level. In various embodiments, the comparison module can be configured using existing commercially-available or freely-available software for comparison purpose, and may be optimized for particular data comparisons that are conducted.

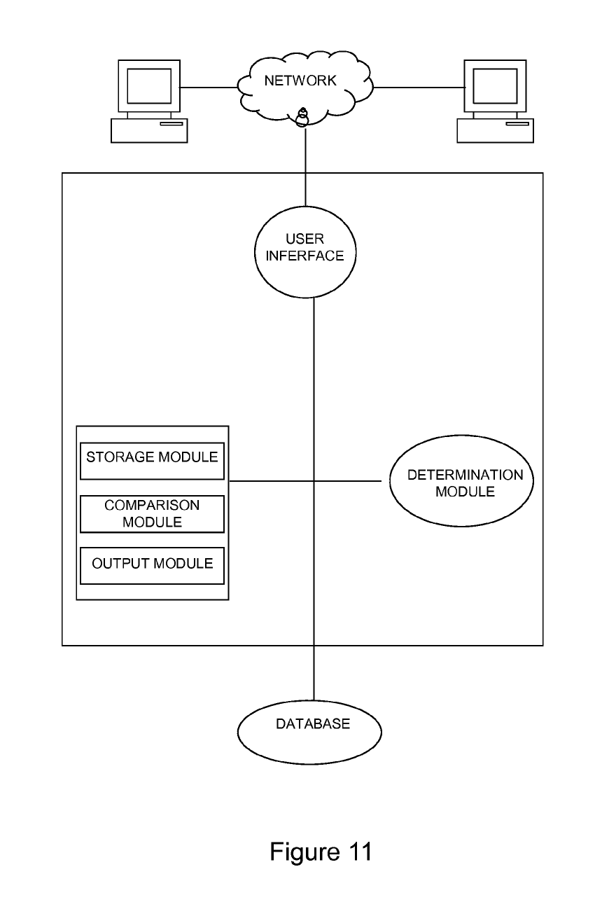

The computing and/or comparison module, or any other module of the invention, can include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). In some embodiments users can directly access data (via Hypertext links for example) residing on Internet databases using a HTML interface provided by Web browsers and Web servers (FIG. 11).

The computing and/or comparison module provides a computer readable comparison result that can be processed in computer readable form by predefined criteria, or criteria defined by a user, to provide content based in part on the comparison result that may be stored and output as requested by a user using an output module, e.g., a display module.

In some embodiments, the content displayed on the display module can be a report, e.g. the level of platelet-adherent leukocytes in the sample obtained from a subject. In some embodiments, a report can denote the level of platelet-adherent leukocytes. In some embodiments, the report can denote raw values of the number/level of platelet-adherent leukocytes in the test sample (plus, optionally, the number/level of platelet-adherent leukocytes in a reference sample) or it indicates a percentage or fold increase in platelet-adherent leukocytes as compared to a reference level, and/or provides a signal that the subject is at risk of having, or not having AERD.

In some embodiments, if the computing module determines that the level of platelet-bound leukocytes in a population of leukocytes in the sample obtained from a subject is greater by a statistically significant amount than the reference level, the display module provides a report displaying a signal indicating that the level in the sample obtained from a subject is greater than that of the reference level. In some embodiments, the content displayed on the display module or report can be the relative level of platelet-adherent leukocytes in the sample obtained from a subject as compared to the reference level. In some embodiments, the signal can indicate the degree to which the level of platelet-bound leukocytes in a population of leukocytes in the sample obtained from the subject varies from the reference level. In some embodiments, the signal can indicate that the subject is at increased risk of having aspirin-exacerbated respiratory disease (AERD). In some embodiments, the signal can indicate the subject can benefit from treatment with a therapy for AERD. In some embodiments, the signal can indicate the subject should not be administered a cyclooxygenase-1 (COX1) inhibitor. In some embodiments, the content displayed on the display module or report can be a numerical value indicating one of these risks or probabilities. In such embodiments, the probability can be expressed in percentages or a fraction. For example, higher percentage or a fraction closer to 1 indicates a higher likelihood of a subject having AERD. In some embodiments, the content displayed on the display module or report can be single word or phrases to qualitatively indicate a risk or probability. For example, a word “unlikely” can be used to indicate a lower risk for having or developing AERD, while “likely” can be used to indicate a high risk for having or developing AERD.

In one embodiment of the invention, the content based on the computing and/or comparison result is displayed on a computer monitor. In one embodiment of the invention, the content based on the computing and/or comparison result is displayed through printable media. The display module can be any suitable device configured to receive from a computer and display computer readable information to a user. Non-limiting examples include, for example, general-purpose computers such as those based on Intel PENTIUM-type processor, Motorola PowerPC, Sun U1traSPARC, Hewlett-Packard PA-RISC processors, any of a variety of processors available from Advanced Micro Devices (AMD) of Sunnyvale, Calif., or any other type of processor, visual display devices such as flat panel displays, cathode ray tubes and the like, as well as computer printers of various types.

In one embodiment, a World Wide Web browser is used for providing a user interface for display of the content based on the computing/comparison result. It should be understood that other modules of the invention can be adapted to have a web browser interface. Through the Web browser, a user can construct requests for retrieving data from the computing/comparison module. Thus, the user will typically point and click to user interface elements such as buttons, pull down menus, scroll bars and the like conventionally employed in graphical user interfaces.

Systems and computer readable media described herein are merely illustrative embodiments of the invention for determining the level of platelet-adherent leukocytes in a sample obtained from a subject, and therefore are not intended to limit the scope of the invention. Variations of the systems and computer readable media described herein are possible and are intended to fall within the scope of the invention. The modules of the machine, or those used in the computer readable medium, may assume numerous configurations. For example, function may be provided on a single machine or distributed over multiple machines.

In some embodiments, described herein is a kit comprising at least one platelet-specific antibody reagent comprising a detectable label. In some embodiments, described herein is a kit comprising at least one leukocyte-specific antibody reagent comprising a detectable label. In some embodiments, described herein is a kit comprising at least one platelet-specific antibody reagent and at least one leukocyte-specific antibody reagent, wherein at least one of the antibody reagents is detectably labeled. In some embodiments, at least one antibody reagent can be immobilized on a solid support. In some embodiments, a kit can further comprise reagents for generating and/or detecting a signal from a detectable label.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:
1. An assay comprising:
   contacting a test sample from a subject with a platelet-specific reagent;
   measuring the percentage of leukocytes to which the reagent is bound;
   wherein the binding of the platelet-specific reagent to a leukocyte indicates the presence of a platelet bound to the leukocyte;
   wherein an increased level of platelet-bound leukocytes relative to a reference level indicates the subject has an increased risk of having aspirin-exacerbated respiratory disease (AERD).
2. An assay comprising:
   (a) contacting the a test sample comprising leukocytes from the peripheral blood of a subject with a platelet-specific antibody reagent and a leukocyte-specific antibody reagent; and
   (b) detecting the presence or intensity of a detectable signal associated with individual cells of the sample using flow cytometry;
   wherein the antibody reagents comprise a detectable label or a means of generating a detectable signal;
   wherein the binding of the platelet-specific antibody reagent to a leukocyte indicates the presence of a platelet-bound leukocyte;
   wherein the binding of the platelet-specific antibody reagent to a leukocyte is determined by the colocalization and/or concurrent detection of the platelet-specific antibody reagent and the leukocyte-specific antibody reagent; and
   wherein an increased level platelet-bound leukocytes within the population of leukocytes, as indicated by the detectable signals, relative to a reference level indicates the subject has an increased risk of having aspirin-exacerbated respiratory disease (AERD).
3. An assay to determine if a subject with a respiratory disease will benefit from treatment with an aspirin-exacerbated respiratory disease (AERD) therapy selected from the group consisting of:
   aspirin desensitization and high-dose aspirin therapy; a P2Y12 inhibitor; montelukast;
   a thromboxane receptor antagonist; a 5-lipoxygenase inhibitor; and zileuton;
   the assay comprising:
   contacting a test sample obtained from the subject with a platelet-specific reagent;
   measuring the percentage of leukocytes to which the reagent is bound;
   wherein the binding of the platelet-specific reagent to a leukocyte indicates the presence of a platelet bound to the leukocyte;
   wherein the binding of the platelet-specific antibody reagent to a leukocyte is determined by the colocalization and/or concurrent detection of the platelet-specific antibody reagent and the leukocyte-specific antibody reagent; and
   wherein an increased level of platelet-bound leukocytes relative to a reference level indicates the subject will benefit from treatment with an AERD therapy.
4. An assay to determine if a subject with a respiratory disease should not be administered a cyclooxygenase-1 (COX1) inhibitor. the assay comprising:
   contacting a test sample obtained from the subject with a platelet-specific reagent;
   measuring the percentage of leukocytes to which the reagent is bound;
   wherein the binding of the platelet-specific reagent to a leukocyte indicates the presence of a platelet bound to the leukocyte; and
   wherein an increased level of platelet-bound leukocytes relative to a reference level indicates the subject should not be administered a COX1 inhibitor.
5. The assay of paragraph 4, wherein the cyclooxygenase-1 (COX1) inhibitor is selected from the group consisting of:
   aspirin; diclofenac; ibuprofen; naproxen; mefenamic acid; indomethacin; ketoprofen; piroxicam; diflunisal; salsalate; dexibuprofen; fenoprofen; dexketoprofen; flurbiprofen; oxaprozin; loxoprofen; indomethacin; sulindac; etodolac; ketorolac; nabumetone; meloxicam; tenoxicam; droxicam; lornoxicam; isoxicam; mefenamic acid; meclofenamic acid; flufenamic acid; and tolfenamic acid.
6. The assay of any of paragraphs 1-5, wherein the platelet-specific reagent is selected from the group consisting of:
   a CD61-binding reagent and a CD41-binding reagent.
7. The assay of any of paragraphs 1-6, wherein the platelets are CD61+ cells.
8. The assay of any of paragraphs 1-6, wherein the platelets are CD41+ cells.
9. The assay of any of paragraphs 1-8, wherein the leukocytes are CD45⁺ cells.
10. The assay of any of paragraphs 2-9, wherein leukocyte-specific antibody reagent is an anti-CD45 antibody reagent.
11. The assay of any of paragraphs 1-10, wherein the leukocytes are neutrophils.
12. The assay of paragraph 11, wherein the neutrophils are CD16⁺ cells.
13. The assay of any of paragraphs 1-10, wherein the leukocytes are eosinophils.
14. The assay of paragraph 13, wherein the eosinophils are CCR3⁺ cells.
15. The assay of any of paragraphs 1-14, wherein the sample comprises a biological tissue selected from the group consisting of:
   whole blood; peripheral blood; whole peripheral blood; a nasal polyp; and products thereof
16. The assay of any of paragraphs 1-15, wherein the level of platelet-bound leukocytes is measured by flow cytometry.
17. The assay of any of paragraphs 1-15, wherein the level of platelet-bound leukocytes is measured by immunocytological methods.
18. The assay of any of paragraphs 1-17, wherein the platelet-specific antibody reagent comprises an antibody or a polypeptide comprising an antigen-binding domain of an antibody.
19. The assay of any of paragraphs 1-18, wherein the subject has increased level of platelet-bound leukocytes relative to a reference level if the level of platelet-bound leukocytes is at least 2× that of the reference level.
20. The assay of any of paragraphs 1-18, wherein the subject has increased level of platelet-bound leukocytes relative to a reference level if the level of platelet-bound leukocytes is at least 3× that of the reference level.
21. The assay of any of paragraphs 1-18, wherein the subject has increased level of platelet-bound leukocytes relative to a reference level if at least 15% of the leukocytes are platelet-bound leukocytes.
22. The assay of any of paragraphs 1-18, wherein the subject has increased level of platelet-bound leukocytes relative to a reference level if at least 50% of the eosinophils are platelet-bound eosinophils.
23. The assay of any of paragraphs 1-18, wherein the subject has increased level of platelet-bound leukocytes relative to a reference level if at least 25% of the neutrophils are platelet-bound neutrophils.
24. The assay of any of paragraphs 1-23, wherein the reference level of platelet-bound leukocytes is the level of platelet-bound leukocytes in a healthy subject without a respiratory disease.
25. The assay of any of paragraphs 1-23, wherein the reference level of platelet-bound leukocytes is the level of platelet-bound leukocytes in a subject with aspirin-tolerant asthma.
26. The assay of any of paragraphs 1-25, wherein the subject is a human.
27. The assay of any of paragraphs 1-26, further comprising creating a report based on the level of platelet-bound leukocytes.
28. A method of administering a treatment for a subject with a respiratory disease, the method comprising:
   contacting a test sample from the subject with a platelet-specific antibody reagent;
   measuring the percentage of leukocytes to which the reagent is bound;
   wherein the binding of the reagent to a leukocyte indicates that the leukocyte is a platelet-bound leukocyte; and
   administering a therapy for aspirin-exacerbated respiratory disease (AERD) if the level of platelet-bound leukocytes is increased relative to a reference level;
   wherein the therapy for AERD is selected from the group consisting of:
   aspirin desensitization and high-dose aspirin therapy; a P2Y12 inhibitor; montelukast; a thromboxane receptor antagonist; a 5-lipoxygenase inhibitor; and zileuton.
29. A method of identifying a subject with a respiratory disease who will benefit from treatment a therapy for aspirin-exacerbated respiratory disease (AERD), the method comprising:
   contacting a test sample from the subject with a platelet-specific antibody reagent; and
   measuring the percentage of leukocytes to which the reagent is bound;
   wherein the binding of the reagent to a leukocyte indicates that the leukocyte is a platelet-bound leukocyte;
   wherein the subject is identified as needing treatment with a therapy for AERD if the level of platelet-bound leukocytes is increased relative to a reference level;
   wherein the therapy for AERD is selected from the group consisting of:
      aspirin desensitization and high-dose aspirin therapy; a P2Y12 inhibitor; montelukast; a thromboxane receptor antagonist; a 5-lipoxygenase inhibitor; and zileuton.
30. A method of determining if a subject is at increased risk of having aspirin-exacerbated respiratory disease (AERD), the method comprising:
   measuring the percentage of platelet-bound leukocytes in a sample obtained from the subject;
   wherein an increased level of platelet-bound leukocytes relative to a reference level indicates the subject is at increased risk of having AERD.
31. The method of any of paragraphs 28-30, wherein the platelet-specific reagent is selected from the group consisting of:
   a CD61-binding reagent and a CD41-binding reagent.
32. The method of any of paragraphs 28-31, wherein the platelets are CD61+ cells.
33. The method of any of paragraphs 28-31, wherein the platelets are CD41+ cells.
34. The method of any of paragraphs 28-33, wherein the leukocytes are CD45⁺ cells.
35. The method of any of paragraphs 28-34, further comprising identifying leukocytes with an anti-CD45 antibody reagent.
36. The method of any of paragraphs 28-35, wherein the leukocytes are neutrophils.
37. The method of paragraph 36, wherein the neutrophils are CD16⁺ cells.
38. The method of any of paragraphs 28-35, wherein the leukocytes are eosinophils.
39. The method of paragraph 38, wherein the eosinophils are CCR3⁺ cells.
40. The method of any of paragraphs 28-39, wherein the sample comprises a biological tissue selected from the group consisting of:
   whole blood; peripheral blood; whole peripheral blood; a nasal polyp; and products thereof 41. The method of any of paragraphs 28-40, wherein the level of platelet-bound leukocytes is measured by flow cytometry.
42. The method of any of paragraphs 28-41, wherein the level of platelet-bound leukocytes is measured by immunocytological methods.
43. The method of any of paragraphs 24-42, wherein the platelet-specific antibody reagent comprises an antibody or a polypeptide comprising an antigen-binding domain of an antibody.
44. The method of any of paragraphs 28-43, wherein the subject has increased level of platelet-bound leukocytes relative to a reference level if the level of platelet-bound leukocytes is at least 2× that of the reference level.
45. The method of any of paragraphs 28-44, wherein the subject has increased level of platelet-bound leukocytes relative to a reference level if the level of platelet-bound leukocytes is at least 3× that of the reference level.
46. The method of any of paragraphs 28-45, wherein the subject has increased level of platelet-bound leukocytes relative to a reference level if at least 15% of the leukocytes are platelet-bound leukocytes.
47. The method of any of paragraphs 28-46, wherein the subject has increased level of platelet-bound leukocytes relative to a reference level if at least 50% of the eosinophils are platelet-bound eosinophils.
48. The method of any of paragraphs 28-47, wherein the subject has increased level of platelet-bound leukocytes relative to a reference level if at least 25% of the neutrophils are platelet-bound neutrophils.
49. The method of any of paragraphs 28-48, wherein the reference level of platelet-bound leukocytes is the level of platelet-bound leukocytes in a healthy subject without a respiratory disease.
50. The method of any of paragraphs 28-48, wherein the reference level of platelet-bound leukocytes is the level of platelet-bound leukocytes in a subject with aspirin-tolerant asthma.
51. The method of any of paragraphs 28-50, wherein the subject is a human.
52. The method of any of paragraphs 28-51, further comprising creating a report based on the level of platelet-bound leukocytes.
53. A computer system for determining if subject has an increased risk of having aspirin-exacerbated respiratory disease (AERD), the system comprising:
   a measuring module configured to measure the level of platelet-bound leukocytes in a population of leukocytes in a test sample obtained from a subject;
   a storage module configured to store output data from the measuring module;
   a comparison module adapted to compare the data stored on the storage module with a reference level, and to provide a retrieved content, and
   a display module for displaying whether the level of platelet-bound leukocytes in a population of leukocytes in a test sample obtained from a subject is greater, by a statistically significant amount, than the reference level and/or displaying the relative levels of platelet-bound leukocytes in a population of leukocytes.
54. The system of paragraph 53, wherein the measuring module measures the presence or intensity of a detectable signal from an immunoassay indicating the presence of platelet-specific antibody reagent on the cells in the test sample.
55. The system of any of paragraphs 53-54, wherein if the computing module determines that the level of platelet-bound leukocytes in a population of leukocytes in the test sample obtained from a subject is greater by a statistically significant amount than the reference level, the display module displays a signal indicating that the level in the sample obtained from a subject is greater than that of the reference level.
56. The system of any of paragraphs 53-55, wherein the signal indicates that the subject is at increased risk of having aspirin-exacerbated respiratory disease (AERD).
57. The system of any of paragraphs 53-56, wherein the signal indicates the subject can benefit from treatment with a wherein the therapy for AERD is selected from the group consisting of:
   aspirin desensitization and high-dose aspirin therapy; a P2Y12 inhibitor;
montelukast; a thromboxane receptor antagonist; a 5-lipoxygenase inhibitor; and zileuton.
58. The system of any of paragraphs 53-57, wherein the signal indicates the subject should not be administered a cyclooxygenase-1 (COX1) inhibitor.
59. The system of any of paragraphs 53-58, wherein the signal indicates the degree to which the level of platelet-bound leukocytes in a population of leukocytes in the sample obtained from the subject vary from the reference level.
60. The assay of any of paragraphs 53-59, wherein the platelet-specific reagent is selected from the group consisting of:
   a CD61-binding reagent and a CD41-binding reagent.
61. The system of any of paragraphs 53-60, wherein the platelets are CD61+ cells.
62. The system of any of paragraphs 53-60, wherein the platelets are CD41+ cells.
63. The system of any of paragraphs 53-62, wherein the leukocytes are CD45⁺ cells.
64. The system of any of paragraphs 53-63, wherein the leukocytes are neutrophils.
65. The system of paragraph 64, wherein the neutrophils are CD16⁺ cells.
66. The system of any of paragraphs 53-62, wherein the leukocytes are eosinophils.
67. The system of paragraph 66, wherein the eosinophils are CCR3⁺ cells.
68. The system of any of paragraphs 53-67, wherein the sample comprises a biological tissue selected from the group consisting of:
   whole blood; peripheral blood; whole peripheral blood; a nasal polyp; and products thereof
69. The system of any of paragraphs 53-68, wherein the level of platelet-bound leukocytes is measured by flow cytometry.
70. The system of any of paragraphs 53-69, wherein the level of platelet-bound leukocytes is measured by immunocytological methods.
71. The system of any of paragraphs 53-70, wherein the platelet-specific antibody reagent comprises an antibody or a polypeptide comprising an antigen-binding domain of an antibody.
72. The system of any of paragraphs 53-71, wherein the subject has increased level of platelet-bound leukocytes relative to a reference level if the level of platelet-bound leukocytes is at least 2× that of the reference level.
73. The system of any of paragraphs 53-72, wherein the subject has increased level of platelet-bound leukocytes relative to a reference level if the level of platelet-bound leukocytes is at least 3× that of the reference level.
74. The system of any of paragraphs 53-73, wherein the subject has increased level of platelet-bound leukocytes relative to a reference level if at least 15% of the leukocytes are platelet-bound leukocytes.
75. The system of any of paragraphs 53-74, wherein the subject has increased level of platelet-bound leukocytes relative to a reference level if at least 15% of the eosinophils are platelet-bound eosinophils.
76. The system of any of paragraphs 53-75, wherein the subject has increased level of platelet-bound leukocytes relative to a reference level if at least 25% of the neutrophils are platelet-bound neutrophils.
77. The system of any of paragraphs 53-76, wherein the reference level of platelet-bound leukocytes is the level of platelet-bound leukocytes in a healthy subject without a respiratory disease.
78. The system of any of paragraphs 53-77, wherein the reference level of platelet-bound leukocytes is the level of platelet-bound leukocytes in a subject with aspirin-tolerant asthma.
79. The system of any of paragraphs 53-78, wherein the subject is a human.
80. The system of any of paragraphs 53-79, further comprising creating a report based on the level of platelet-bound leukocytes.
81. A method of directing the treatment of a subject in need of treatment for AERD, the method comprising:
   a) measuring a first level of platelet-bound leukocytes in a sample obtained from a subject as described herein;
   b) administering a treatment for AERD if the subject is determined to have an increased level of platelet-bound leukocytes relative to a reference;
   c) measuring a second level of platelet-bound leukocytes in a sample obtained from a subject as described herein; and
   d) adjusting the dosage of the treatment as indicated by the second level of platelet-bound leukocytes.
82. The method of paragraph 81, wherein the treatment is selected from the group consisting of:
   a P2Y12 inhibitor; a leukotriene receptor antagonist; montelukast; a thromboxane receptor antagonist; a 5-lipoxygenase inhibitor; and zileuton

### EXAMPLES

### Example 1

Cysteinylleukotriene (cysLT) overproduction is a hallmark of aspirin-exacerbated respiratory disease (AERD), but its mechanism is poorly understood. Because adherent platelets can convert the leukocyte-derived precursor leukotriene (LT)A₄ to LTC₄, the parent cysLT, through the terminal enzyme LTC₄ synthase, the contribution of platelet-dependent transcellular cysLT production in AERD was investigated. Nasal polyps from subjects with AERD contained many extravascular platelets that colocalized with leukocytes, and the percentages of circulating neutrophils, eosinophils, and monocytes with adherent platelets were markedly higher in the blood of subjects with AERD than in aspirin tolerant controls. Platelet-adherent subsets of leukocytes had higher expression of several adhesion markers than did platelet nonadherent subsets. Adherent platelets contributed more than half of the total LTC₄ synthase activity of peripheral blood granulocytes, and they accounted for the higher level of LTC₄ generation by activated granulocytes from subjects with AERD compared with aspirin tolerant controls. Urinary LTE₄ levels, a measure of systemic cysLT production, correlated strongly with percentages of circulating platelet-adherent granulocytes. Because platelet adherence to leukocytes allows for both firm adhesion to endothelial cells and augmented transcellular conversion of leukotrienes, a disturbance in platelet-leukocyte interactions may be partly responsible for the respiratory tissue inflammation and the overproduction of cysLTs that characterize AERD. (Blood. 2012; 119(16):3790-3798).

Aspirin-exacerbated respiratory disease (AERD) is a distinctive syndrome characterized clinically by a triad of asthma, nasal polyposis, and aspirin sensitivity. It is a chronic inflammatory disease associated with eosinophilic infiltration of respiratory tissues, peripheral eosinophilia, and excessive production of cysteinyl leukotrienes (cysLTs), a class of inflammatory lipid mediators that are thought to contribute to several of the characteristic features of AERD. Individuals with this syndrome account for 4% to 11% of all adult patients with asthma, and for a disproportionate share (˜30%) of patients with severe asthma.¹ The confirmatory diagnostic feature of AERD is an idiosyncratic respiratory reaction, including symptoms of acute bronchoconstriction, nasal congestion, and eye watering, on ingestion of aspirin or another nonselective cyclooxygenase (COX) inhibitor. Despite the strikingly consistent clinical phenotype of AERD, the pathogenesis of the disease remains unclear.

CysLTs derive from the metabolism of arachidonic acid by effector cells of the innate immune system. In inflammatory leukocytes (neutrophils, monocytes, eosinophils, mast cells, and basophils), arachidonic acid is oxidized by 5-lipoxygenase (5-LO) to form the unstable intermediate leukotriene (LT)A₄.² In neutrophils, LTA₄ is preferentially hydrolyzed by LTA₄ hydrolase to form LTB₄, whereas in monocytes, mast cells, eosinophils, and basophils, it is conjugated to reduced glutathione by the terminal enzyme LTC₄ synthase (LTC₄S) to form LTC₄, the parent cysLT.³ LTC₄ is exported out of the cell and enzymatically converted into LTD₄ and then into the stable end-metabolite LTE₄. Urinary LTE₄ levels, a marker of systemic cysLT production, are 3 to 5 times higher in patients with AERD than in their aspirin-tolerant counterparts at baseline, and these levels can further increase by as much as 100-fold on ingestion of aspirin.⁴ LTC₄ and LTD₄ are powerful smooth muscle constrictors,⁵ LTE₄ potently induces the accumulation of eosinophils into the bronchial mucosa,⁶ and all 3 cysLTs can induce vascular leak, mucous production, edema, and fibrosis. Thus, cysLTs contribute to the chronic inflammation present in the respiratory tissue of patients with AERD. They are also critical effectors of the aspirin-induced reactions that characterize AERD, because both inhibition of 5-LO and blockade of the type 1 receptor for cysLTs (CysLT receptor) can blunt the clinical severity of symptoms occurring with aspirin challenges.^{7,8} However, neither the cellular source of cysLTs nor the mechanisms for their overproduction in AERD are known. Because eosinophils, basophils, mast cells, and macrophages express both 5-LO and LTC₄S, they are able to catalyze the formation of LTC₄ from endogenous arachidonic acid, and they probably contribute to the production of cysLTs in AERD. However, apart from immunohistochemical studies suggesting that eosinophils in the respiratory tract mucosa express higher levels of LTC₄S protein in subjects with AERD than in aspirin-tolerant controls, no abnormalities in cysLT generation have been reported in cells from individuals with AERD.^{9,10}

Moreover, it is not understood how the 5-LO-derived substrate LTA₄ is provided at sufficient quantities to permit the high basal production of cysLTs characteristic of AERD. Of the circulating cells possessing 5-LO activity, neutrophils are by far the most plentiful and can generate quantities of LTA₄ that exceed the capacity of their LTA₄ hydrolase to convert it into LTB₄. Although neutrophils lack LTC₄S activity and cannot convert LTA₄ into LTC₄, platelets possess abundant LTC₄S activity in the absence of 5-LO.^{11,12} Previous ex vivo studies have shown that platelets can convert unmetabolized LTA₄ from neutrophils or monocytes into LTC₄ through a transcellular pathway that requires P-selecting dependent interaction between the platelet and the leukocyte.¹³⁻¹⁵

Allergen-induced pulmonary eosinophilia and airway remodeling in mouse models of asthma also require P-selecting dependent adherence of platelets to leukocytes and subsequent augmentation of leukocyte integrin function.¹⁶ Because AERD involves both accumulation of leukocytes, particularly eosinophils, in the respiratory tissue, and systemic overproduction of cysLTs, it was hypothesized that platelet-leukocyte interactions can contribute to this disease. As described herein, the frequencies of platelet-adherent leukocytes in the sinus tissue and blood of subjects with AERD was determined and compared to those found in the tissue and blood of aspirin-tolerant controls. Also described herein are experiments directed to whether adherent platelets contributed to the activation of leukocytes and to the production of cysLTs in vivo and in vitro.

### Methods

Patients, Materials, and Human Subject Characterization.

Patients were recruited from the Allergy, Pulmonary, and Otolaryngology clinics at the Brigham and Women's Hospital (Boston, Mass.) and were classified according to their clinical characteristics. Nonasthmatic controls had no history of asthma or intolerance to aspirin or other nonsteroidal anti-inflammatory drugs. Aspirin-tolerant asthmatic (ATA) controls had physician-diagnosed persistent asthma and had taken aspirin or a nonsteroidal anti-inflammatory drug within the previous 6 months without adverse reaction. Patients were suspected of having AERD if they had asthma, nasal polyposis, and a history of respiratory reaction on ingestion of a COX inhibitor. In all subjects with a compatible clinical history, the diagnosis of AERD was confirmed with a graded oral challenge to aspirin that resulted in characteristic sinonasal symptoms and a decrease in forced expiratory volume in 1 second of at least 15%. None of the subjects smoked. All subjects with AERD were treated with the CysLT₁ receptor blocker montelukast during the aspirin challenge, and none were on the 5-LO inhibitor zileuton before the challenge. Clinical data regarding the subjects including pulmonary function, presence of atopy (defined as 2 or more positive skin prick tests), and use of corticosteroids and long-acting β-agonists are summarized in Table 1. For the immunohistochemical studies, nasal polyps were collected after their surgical excision from subjects with AERD or from aspirin-tolerant controls with chronic hyperplastic sinusitis. These controls with sinusitis were judged to be aspirin-tolerant if they had taken aspirin or a nonsteroidal anti-inflammatory drug within the previous 6 months without adverse reaction; 2 of the 4 aspirin tolerant controls also had asthma. All subjects had been treated with oral prednisone (20 mg daily) for the week leading up to their sinus surgery. The Brigham and Women's Hospital institutional human subjects Institutional Review Board (protocol 2003-P-002088) approved the study, and all subjects provided written consent in accordance with the Declaration of Helsinki.

Immunohistochemistry.

For the nasal polyp studies, polyp tissue was excised at the time of surgery, placed in sterile normal saline, and grossly examined by the pathology department. Half of the fresh tissue was allocated for experimentation and within 2 hours of surgery the specimens were fixed in 4% paraformaldehyde, embedded in TISSUE-TEK O.C.T. COMPOUND™ (Sakura Finetek), and kept at −80° C. until sectioning. For immunofluorescent detection of leukocytes and platelets, frozen sections were air-dried, blocked with 10% mouse serum, cut into 8-μm slices, and incubated for 1 hour at room temperature with ALEXA FLUOR™ 488-labeled anti-CD45 (4 μg/mL) and ALEXA FLUOR™ 647-labeled anti-CD61 (4 μg/mL) Abs (BioLegend). Slides were then washed with PBS and mounted with SLOWFADE GOLD™ antifade reagent with 4,6 diamidino-2-phenylindole nuclear stain (Invitrogen). The dry sections were evaluated under an 80i microscope (Nikon); photographs were taken under 40× objective lens (Panfluor 40×, aperture 0.75), with pictures taken for red, green, and blue wavelengths, and overlaid into RGB pictures using IMAGEJ™ (Version 1.71) software (National Institutes of Health). At least 4 randomly selected fields from each tissue sample were photographed using a Hamatsu ORCA™ R2 digital camera (C10600). Images were acquired with HC IMAGE™ software (Version 2.0.4) and evaluated by a pathologist who was blinded as to the subject's diagnosis. Cells staining for CD45 (green) and for both CD45 and CD61 (red) were counted. Separate sections were used for H&E staining.

Flow Cytometry.

Whole peripheral blood was drawn into heparinized tubes, kept at room temperature, and assayed within 1 hour of collection. For subjects undergoing oral aspirin challenge, blood was collected before ingestion of aspirin. Ten μL, of unstimulated blood was incubated with directly conjugated antibodies specific for CD61 and CD45, and CCR3, CD11a, CD11b, CD11c, CD16, CD18, P-selectin, and/or P-selectin glycoprotein ligand 1 (PSGL-1), or appropriate isotype controls (BD Biosciences) for 20 minutes, and then fixed the cells in 1% paraformaldehyde. At least 20,000 CD45⁺ cells were recorded for each sample on an FACSARIA™ flow cytometer (BD Biosciences), and they were analyzed with FLOWJO™ Version 7.6.4 (TreeStar). CD45⁺ leukocytes were classified as eosinophils, neutrophils, monocytes, or lymphocytes according to their side scatter characteristics and relative expression of CD45, CD16 (to identify neutrophils), and CCR3 (to identify eosinophils), and they were assessed for the presence of adherent platelets by relative expression of CD61. Within each leukocyte population, the mean fluorescence intensity of each activation or adhesion marker was measured separately for the platelet-adherent subset and the platelet-free subset.

Western Blot Analysis.

To measure platelet LTC₄S protein, washed platelets were separated from whole blood by centrifugation, and 10 μg of platelet protein lysate was used to generate gels and then transferred onto IMMUN-BLOT™ polyvinylidene difluoride membranes (Bio-Rad Laboratories) and blocked with 5% milk in tris(hydroxymethyl)aminomethane-buffered saline. Blots were incubated with either a polyclonal anti-LTC₄S antibody¹² or an anti-β-actin antibody (Cell Signaling Technology), washed, and then incubated with HRP conjugated anti-rabbit IgG (Sigma-Aldrich) and visualized by enhanced chemiluminescence (GE Healthcare).

Activation of Granulocytes and Analysis of 5-LO Pathway Products and LTC₄S Activity.

Platelet-rich plasma was removed from whole peripheral blood by centrifugation, washed, and resuspended at a concentration of 1×10⁹ platelets/mL. Leukocytes were separated by 4.5% dextran gradient, and the granulocyte fraction was obtained by FICOLL-PAQUE™ (GE Healthcare) density gradient centrifugation. Contaminating erythrocytes were lysed with a hypotonic saline wash. Granulocytes were counted and a portion was stripped of adherent platelets by incubation with 0.05% trypsinethylenediaminetetraacetic acid for 15 minutes at 37° C.¹⁷ Removal of adherent platelets was confirmed by cytofluorographic analysis of samples of the granulocytes before and after trypsinization. Supernatants from unstripped granulocytes and platelet-stripped granulocytes stimulated for 10 minutes with 5 μM A23187 were analyzed using reverse-phase high performance liquid chromatography (RP-HPLC; Beckman Coulter) as described previously.¹⁸ The resolved products, measured from their absorbance at 280 nm, were calculated from the ratio of the peak areas to the area of the internal standard prostaglandin B₂ (PGB2) and included measurement of LTB₄ (retention time, 23.9 minutes), LTC₄ (retention time, 21.8 minutes), LTD₄ (retention time, 23.6 minutes), (5,6)-dihydroxy-7,9-trans-11,14-cis-eicosatetraenoic acid (retention time, 26.1 minute), and 6-trans-LTB₄ (retention time, 23.2 minutes).¹⁹ In some experiments, supernatants from granulocytes stimulated for 10 minutes with 2 μM formyl-methionyl-leucylphenylalanine (fMLP) were analyzed for cysLTs using the Amersham LEUKOTRIENE C4/D4/E4 BIOTRAK™ enzyme immunoassay (GE Healthcare), because cysLT concentrations induced by fMLP are too low to detect by RP-HPLC. In some experiments, aliquots of granulocytes that had been subjected to trypsinization were stimulated with A23187 in the presence of 200×10⁶ exogenously added platelets to verify that their enzyme function was not compromised by trypsinization and could be restored by platelets.

The specific activity of the terminal cysLT-generating enzyme LTC₄S was measured by cellular conversion of exogenous LTA₄-methyl ester (ME) to LTC₄-ME as described using RP-HPLC.³ In brief, aliquots of 200×10⁶ washed platelets or 6×10⁶ granulocytes (with or without removal of platelets by trypsinization) were provided with 10 mM glutathione and 20 μM LTA₄-ME. After 15 minutes at 37° C., the reaction was terminated with methanol containing PGB2. LTC₄-ME was quantified from the ratio of the peak area to the area of the internal standard PGB₂.

Gas Chromatography—Mass Spectrometry.

Urine samples were collected, stored at −80° C., and analyzed by gas chromatography—mass spectrometry as described previously to measure concentrations of LTE₄,²⁰ the major urinary thromboxane metabolite 11-dehydrothromboxane B₂ (TXB2),²¹ and F2-isoprostanes,²² all normalized for creatinine. For subjects undergoing oral aspirin challenge, urine was collected before ingestion of aspirin.

Statistical Analysis.

The data are presented as the mean+SEM unless otherwise stated. Differences in values were analyzed with the t test, because all data presented was normally distributed; significance was defined as P<0.05, and all tests were 2-tailed. Effect size was measured with Pearson correlation coefficient.

### Results

Patient Characteristics.

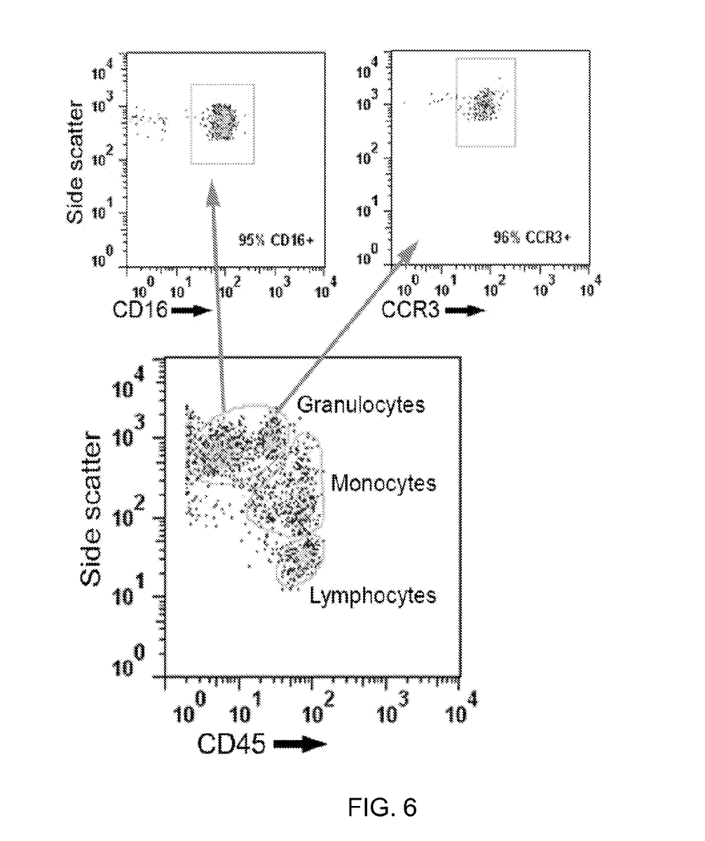

The 3 groups of patients were similar in age and sex, and there were no significant differences between the 2 groups of asthmatic subjects in regard to the baseline forced expiratory volume in 1 second, the presence of atopy, or the proportion of patients receiving daily inhaled corticosteroids or long-acting β-agonists as controller therapies (Table 1). The percentage of circulating granulocytes identified as eosinophils (CCR3⁺ cells within the CD45⁺ granulocyte gate; FIG. 6) did not differ significantly between the 2 groups of asthmatic subjects, although nonasthmatic control subjects had lower percentages of eosinophils than either the ATA controls (P<0.05) or the subjects with AERD (P<0.01).

Identification of Extravasated Platelets and their Colocalization with Leukocytes in Nasal Polyp Tissue.

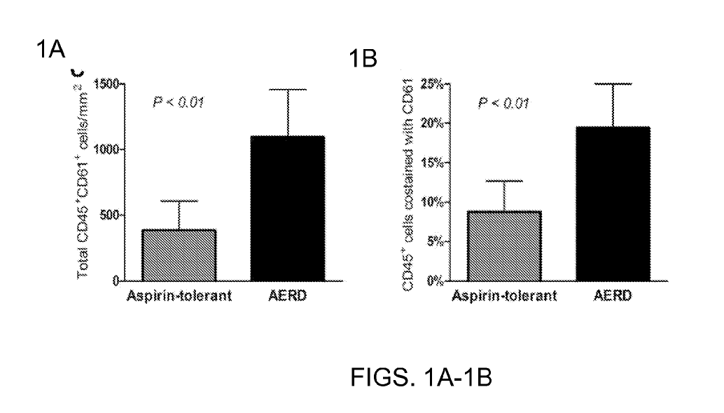

To determine whether platelet-adherent leukocytes were present in the inflamed respiratory tissue of subjects with AERD and of aspirin-tolerant controls with sinusitis, sections from surgically excised nasal polyps were double-stained for the leukocyte-specific antigen CD45 and the platelet-specific antigen CD61 Immunofluorescence was used to identify cells expressing each marker. The tissue from subjects with AERD contained abundant eosinophils, as determined by H&E staining (data not shown) and many extravascular CD61⁺ platelets (data not shown) were present and colocalized with CD45+ leukocytes (data not shown). The total numbers of eosinophils were higher in subjects with AERD (196±203/mm²) than in aspirin-tolerant controls (2±2/mm²), although this difference did not reach statistical significance because of variation within the AERD group. The total numbers of CD45⁺ leukocytes in the tissue did not differ significantly between the groups (6153±2108/mm² in AERD and 4201±860/mm² in ATA). The tissue from subjects with AERD had more total platelet-associated leukocytes and a higher percentage of leukocytes that colocalized with platelets (FIGS. 1A-1B).

Platelet-Adherent Leukocytes are Detected with High Frequency in AERD and Exhibit Altered Expression of Integrins.

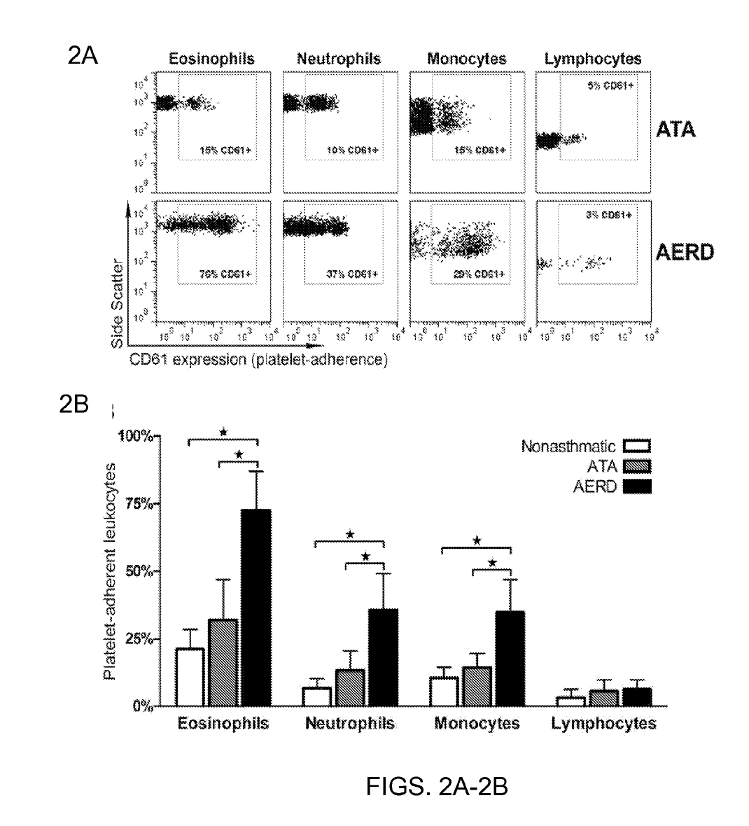

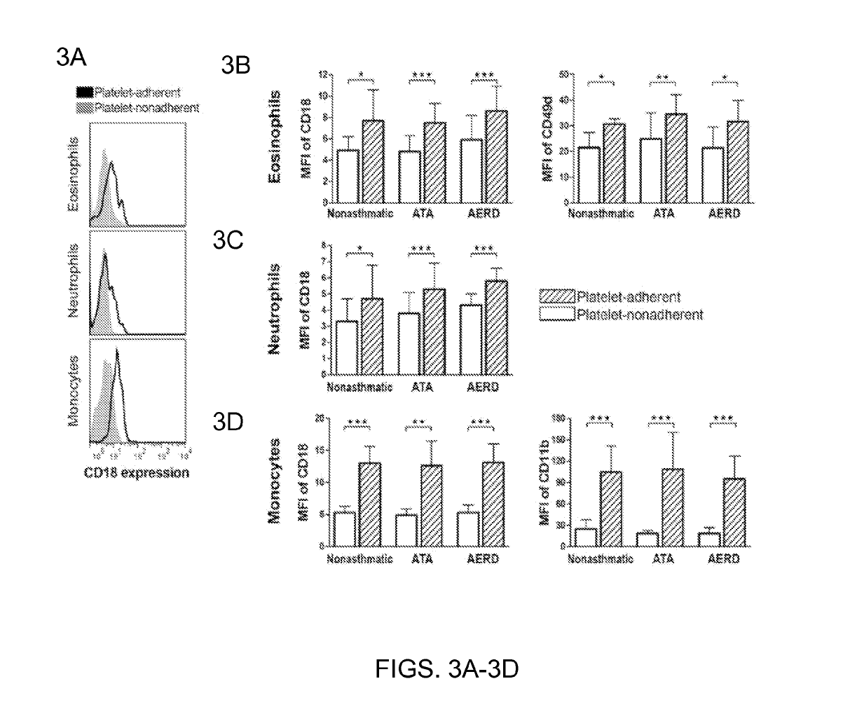

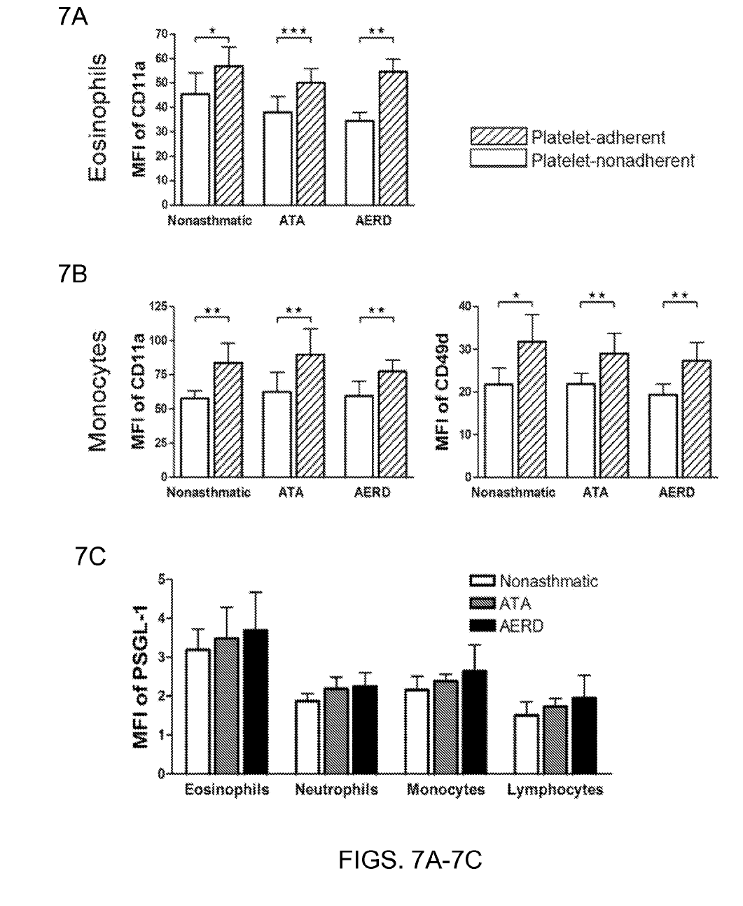

To determine whether platelet-adherent leukocytes could be identified in the peripheral blood, whole blood samples were studied by flow cytometry, using CD45 to identify leukocytes and CD61 to identify platelets. Within the CD45⁺ gate, eosinophils, neutrophils, monocytes, and lymphocytes were distinguished based on differential light scatter characteristics and their relative membrane expressions of CCR3 and CD16 (FIG. 6). Because these cell types also could be readily distinguished from one another based solely on their light scatter characteristics and relative expression of CD45 (FIG. 6), these parameters were used to identify the leukocyte cell types in the experiments in which adhesion receptor expression was quantified. Platelet-adherent leukocytes were identified in all patient groups (FIG. 2A). There tended to be higher percentages of platelet-adherent leukocytes in the blood of ATA controls than in nonasthmatic controls, but these differences were not significant. In contrast, the percentages of platelet-adherent eosinophils, neutrophils, and monocytes in the blood of subjects with AERD were much higher than in either the ATA or the nonasthmatic controls (FIG. 2B). Few platelet-adherent lymphocytes were detected in any group. To determine the effect of adherent platelets on the expression of integrins by leukocytes, the surface expression of adhesion receptors was quantified on platelet-adherent and nonadherent subsets of leukocytes, identified as described in the previous paragraph. In all patient groups, platelet adherence was associated with modest increases in the expression of CD18, CD11a, and CD49d by eosinophils (FIGS. 3A-B and FIG. 7A) and modest increases in the expression of CD18 by neutrophils (FIG. 3A,C).

Platelet-adherent monocytes displayed markedly up-regulated expression of CD18 and CD11b compared with platelet nonadherent monocytes in the same samples (FIGS. 3A-3D), and also showed modestly increased expression of CD49d, CD11a, and CD11c (FIG. 7B; data not shown). All patient groups showed similar patterns, and there were no differences between the patient groups in the expression levels of PSGL-1 on any leukocyte subsets (FIG. 7C) or P-selectin by platelets (data not shown).

Adherent Platelets Contribute to Granulocyte-Associated LTC₄S Activity and cysLT Production by Activated Granulocytes In Vitro.

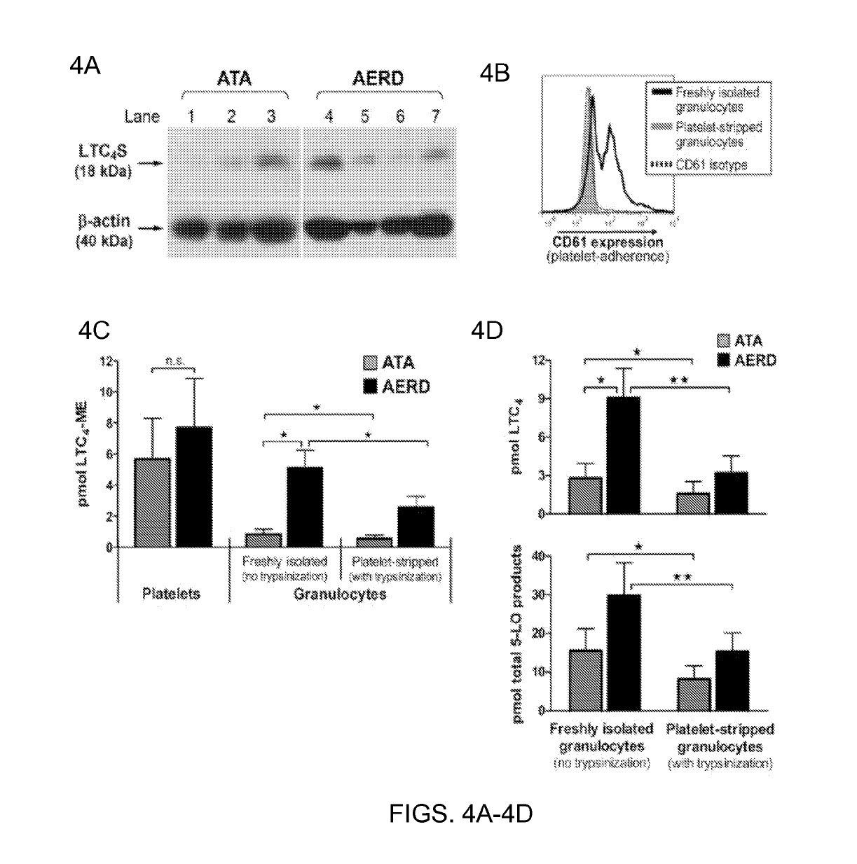

To determine whether platelets could contribute to the production of cysLTs by granulocytes, and whether this contribution differed between subjects with AERD and ATA controls, the expression and function of LTC₄S in platelets from the blood of subjects with AERD and ATA controls was studied. The ability of peripheral blood granulocyte fractions to generate cysLTs and to convert exogenous LTA₄-ME to LTC₄-ME was measured, both before and after the removal of adherent platelets using trypsin. Washed platelets expressed the 18-kDa LTC₄S protein (FIG. 4A), with platelets from both ATA and AERD subjects displaying a range of expression levels. Trypsinization removed more than 90% of the adherent platelets from granulocytes in vitro, as determined by cytofluorographic analysis (representative histogram, FIG. 4B). Platelets from both groups demonstrated specific LTC₄S activity, measured by conversion of exogenous LTA₄-ME to LTC₄-ME (FIG. 4C left), and there were no significant differences in LTC₄S activity between platelets from subjects with AERD and those from controls. However, LTC₄S activity was higher in the freshly isolated granulocytes of subjects with AERD, and activity decreased after trypsinization and stripping of platelets, by 54±12% and 56±3% in ATA and AERD subjects, respectively. The LTC₄S activity of platelet-stripped granulocytes from the subjects with AERD remained higher than those from ATA controls (FIG. 4C right). For the granulocyte samples used in the LTA₄-ME conversion assay, the percentage of granulocytes identified as CCR3⁺ eosinophils was 10.4±7.1% and 13.4±5.2% for ATA and AERD subjects, respectively.

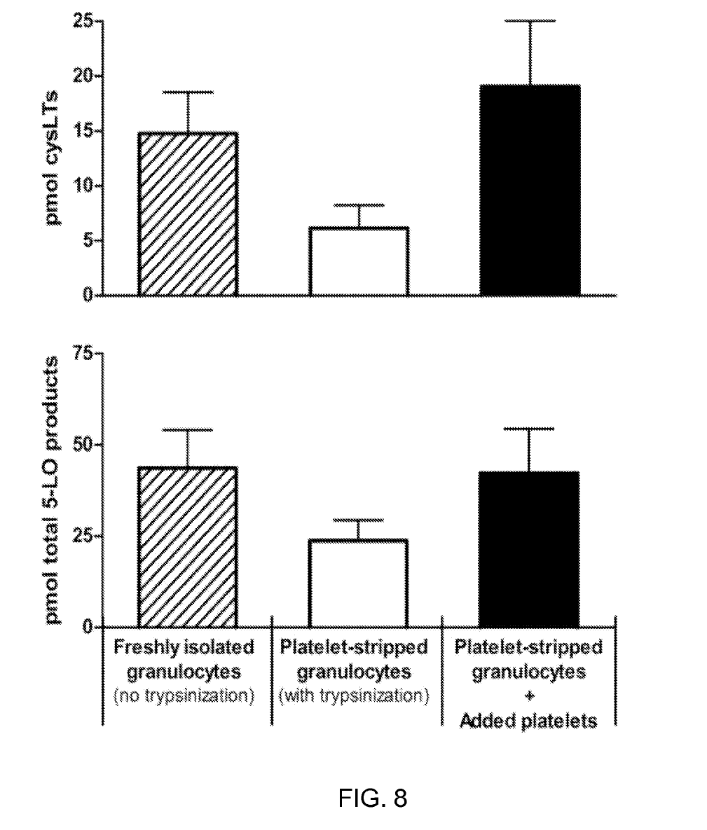

When stimulated with A23187, granulocytes generated the entire spectrum of 5-LO pathway products, including LTB₄, LTC₄, LTD₄, and the LTA₄ hydrolysis metabolites (5,6)-dihydroxy-7,9-trans-11,14-cis-eicosatetraenoic acid and 6-trans-LTB₄. Freshly isolated A23187-stimulated granulocytes from subjects with AERD generated more LTC₄ (FIG. 4D top) and more total 5-LO pathway products (FIG. 4D bottom) than did granulocytes from aspirin tolerant controls. After removal of platelets by trypsinization, the A23187-induced production of LTC₄ specifically decreased by 58±10% and 68±11%, and the production of total 5-LO pathway products decreased by 48±8% and 41±4%, in the granulocytes from ATA and AERD subjects, respectively (FIG. 4D). Removal of platelets also reduced fMLP-induced generation of cysLTs by 64±16% (from 623±203 pg [0.99±0.31 pmol] to 227±129 pg [0.35±0.21 pmol]/mL) in 3 separate experiments using granulocytes from subjects with AERD. The addition of 200×10⁶ autologous platelets to the trypsinized granulocytes restored robust generation of all 5-LO pathway products, including LTC₄, confirming that cell viability and function of the required enzymes were not intrinsically compromised by trypsinization (n=6; FIG. 8).

Platelet-Adherent Granulocytes Correlate with Systemic cysLT Production In Vivo.

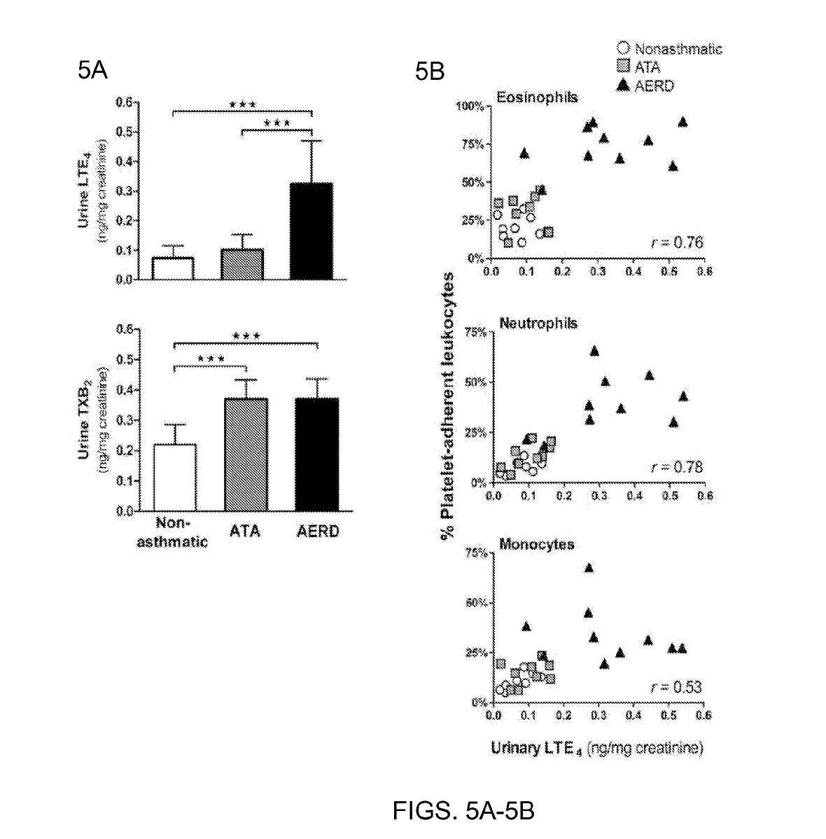

To determine whether platelet-adherent leukocytes contributed to the generation of cysLTs in vivo, baseline urinary LTE₄ levels were measured for each subject. Levels of TXB2, the stable metabolite of thromboxaneA2, the major platelet-derived COX product, were measured in the same samples. As expected, the urine of subjects with AERD had strikingly higher levels of LTE₄ than either aspirin-tolerant control group (FIG. 5A top). Urinary LTE₄ levels correlated strongly with the percentages of platelet-adherent eosinophils and neutrophils and moderately with platelet-adherent monocytes in the peripheral blood (FIG. 5B). Levels of urinary TXB2 levels in both groups of asthmatics were nearly double those of nonasthmatic controls (FIG. 5A bottom), but they did not differ between ATA and AERD subjects and did not correlate with the percentages of circulating platelet-adherent leukocytes (data not shown). Urine from separate cohorts of 4 aspirin-tolerant controls and 9 subjects with AERD also was analyzed for baseline levels of F2-isoprostanes that can act as agonists for platelet activation and arise as a result of endogenous oxidant stress. Isoprostane levels did not differ significantly between the groups (1.2±0.3 and 1.5±0.4 ng/mg creatinine for aspirin-tolerant controls and AERD subjects, respectively; data not shown), and the measurements from both groups were similar to the published normal human values of 1.6±0.6 ng/mg creatinine ²³ Circulating total white blood cell and platelet counts were measured in 11 subjects, and neither cell count was correlated with urinary LTE₄ levels (data not shown).

### Discussion

The data described herein indicate that platelets and platelet-adherent leukocytes are effectors of AERD, a distinctive variant of asthma characterized by idiosyncratic reactions to nonselective COX inhibitors, and marked bronchial and sinonasal tissue eosinophilia. Based on functional and pharmacologic studies, these features of the disease are likely causally related to dysregulated cysLT production, the basis of which has remained evasive. Platelet-leukocyte aggregates are proposed to contribute to vascular inflammation in cardiovascular disease²⁴ and have been identified in the blood of subjects with allergic asthma during late-phase responses to inhaled allergen.²⁵ However, no previous study provides direct evidence of a pathogenic role for platelet-leukocyte aggregates in human disease. The frequencies of platelet-adherent eosinophils, neutrophils, and monocytes in the blood of subjects with AERD are strikingly increased relative to their frequencies in the blood of aspirin-tolerant controls (FIGS. 2A-2B). The enhanced expression of integrin subunits on these platelet-adherent leukocyte subsets (FIG. 3 and FIGS. 7A-7C) supports a previously hypothesized mechanism by which platelets prime leukocyte adhesion to endothelial cells,¹⁶ and suggests that platelets may amplify tissue inflammation in AERD.

This conclusion is further supported by the immunohistochemical data in nasal polyp tissue (FIGS. 1A-1B). Moreover, the correlation between the frequencies of platelet-adherent granulocytes in the blood and baseline urinary levels of LTE₄ (FIGS. 5A-5B), a reflection of systemic cysLT generation, and the substantial contribution from adherent platelets to total 5-LO pathway products and LTC₄S activity in peripheral blood granulocytes (FIGS. 4A-4D), further indicate a causal link.

In addition to their role in hemostasis, platelets are implicated as effectors of leukocyte recruitment and as sources of bioactive mediators in cardiovascular disease and in mouse models of acid-induced lung injury, rheumatoid arthritis, and skin fibrosis.²⁶⁻²⁹ Older studies identified activated platelets in bronchial biopsies from subjects with asthma,³⁰ and platelets are essential to the accumulation of eosinophils and the development of inflammation in lungs of allergen-sensitized and challenged mice.^{16,31} Dramatic tissue eosinophilia characterizes AERD and the experiments described herein found eosiniophilia in all 6 nasal polyps from subjects with AERD despite the fact that all subjects had been treated with systemic corticosteroids for 5 days preoperatively, but very few eosinophils in the polyps from the aspirin tolerant controls, who also received systemic steroids. Extravasated platelets were readily detected in nasal polyps from individuals with AERD, and the numbers and percentages of CD45⁺ leukocytes that colocalized with CD61⁺ platelets in these specimens exceeded the numbers and percentages in the nasal polyps from aspirin-tolerant controls with sinusitis by 2- to 3-fold (FIGS. 1A-1B).

Flow cytometry revealed that the frequencies not only of platelet adherent eosinophils but also of platelet-adherent neutrophils and monocytes were markedly higher in AERD than in the controls (FIGS. 2A-2B). Although eosinophils dominate the inflammatory cell infiltrate typical of the bronchial and sinonasal mucosa in asthma and AERD, neutrophils and macrophages also are increased in number compared with healthy tissues.^{9,32} Taken together, these observations indicate that platelets may adhere to leukocytes as a prelude to their recruitment into the tissues and that AERD may involve a disturbance in the homeostasis that controls this process. Moreover, the tissue eosinophilia in AERD may be relatively resistant to corticosteroids, consistent with the refractory nature of the nasal polyposis in this syndrome.

The previously recognized platelet-dependent pathway for the development of allergen-induced pulmonary inflammation in mice requires binding of platelet-associated P-selectin to leukocyte-associated PSGL-1.¹⁶ This interaction primes leukocytes for adhesion to endothelial cells by up-regulating the expression and avidity of β1- and β2 integrins on the leukocyte membrane and has been demonstrated in eosinophils, neutrophils, and monocytes.³³⁻³⁵ To determine whether this pathway was operative in AERD, the cell-specific effects of platelet adherence on the subsequent surface expression of leukocyte activation and adhesion receptors was studied by separately gating on platelet-adherent and -nonadherent leukocyte subsets in the blood. Especially strong up-regulation of CD18 expression was found in platelet-adherent monocytes (FIGS. 3A-3D), as well as significant increases in CD11a, CD11b, and CD11c (FIGS. 3A-3D and FIGS. 7A-7C), each of which partner with CD18 to form the β2 integrins, which permit firm adhesion of leukocytes to endothelial and epithelial cells via intracellular adhesion molecule-1 (ICAM-1). In monocytes, the up-regulation of CD11b most closely paralleled the increase in CD18, suggesting that platelet adherence may potentiate the CD11b/CD18 (MAC-1)-dependent pathway for monocyte recruitment. CD18 also was modestly increased on both neutrophils and eosinophils that were platelet-adherent (FIGS. 3A-3D), along with CD11a in eosinophils.

Compared with the platelet nonadherent fractions, platelet adherent eosinophils and monocytes also expressed higher levels of CD49d (FIGS. 3A-3D and FIGS. 7A-7C) that pairs with CD29 to form very late antigen-4 integrin that is particularly important for the recruitment of eosinophils, basophils, monocytes, and lymphocytes to sites of allergic inflammation.^{36,37} Although none of the platelet-related changes in leukocyte receptor expression were specific to cells from subjects with AERD, the substantial differences in the total percentages of circulating leukocytes to which platelets adhere in AERD implies that the platelet-dependent effects on adhesion receptor expression may be especially relevant to tissue inflammation in AERD. The mechanism for this increase in AERD remains to be determined but does not seem to reflect a difference in expression levels of either PSGL-1 on leukocytes (FIGS. 7A-7C) or P-selectin on platelets (data not shown).

Although cysLTs play a validated role in asthma,^{38,39} their pathogenic role in AERD is especially prominent because of their overproduction and to the increased function of their receptors.^{4,9,10} In addition to activating eosinophils, mast cells, and monocytes,⁴⁰ aspirin challenges also elicit increases in LTB₄ metabolites in the urine of subjects with AERD that parallel increases in LTE₄,⁴¹ suggesting that dysregulation of 5-LO activity in neutrophils is another feature of AERD. More than 50% of the LTA₄ synthesized by neutrophils is released unmetabolized into the extracellular milieu,⁴² and is only available as a substrate for reuptake by adherent cells because of its extracellular half-life of less than 5 seconds.⁴³ Although ex vivo studies indicate that platelet conversion of neutrophil- or monocyte-derived LTA₄ into LTC₄ requires adherence to leukocytes via P-selectin/PSGL-1, this transcellular pathway had not been demonstrated previously in any human disease.¹³ Because the 3 major 5-LO-expressing cell types in the peripheral blood—neutrophils, eosinophils, and monocytes—all showed increased adherence to platelets in subjects with AERD relative to aspirin-tolerant control subjects, it was suspected that adherent platelets might contribute to the increased cysLT generation that is a signature of AERD.

Platelets expressed the same 18-kDa enzyme that is expressed by eosinophils, mast cells, and monocytes (FIG. 4A). By removing platelets from granulocytes using trypsinization, it was determined that platelets contribute more than half of the total LTC₄S activity (measured using an assay of specific enzymatic activity; FIG. 4C) in freshly isolated peripheral blood granulocytes in AERD, as reflected by the “trypsin-sensitive” component of LTC₄S activity. Although adherent platelets accounted for similar fractions of LTC₄S activity in the granulocytes of the ATA and AERD groups, the increased frequencies of platelet-adherent granulocytes in the samples from subjects with AERD resulted in substantially greater A23187-induced production of LTC₄ from these samples than from the ATA controls (FIG. 4D). The higher level of trypsin-insensitive LTC₄S activity in the granulocytes from subjects with AERD (FIG. 4C) is likely because of slightly higher percentage of eosinophils in those samples, and because of increased LTC₄S activity in eosinophils as suggested by previous immunohistochemical studies.^{9,10} Adherent platelets also accounted for a remarkably similar fraction (64±16%) of the cysLTs produced in response to fMLP, which was chosen as a physiologic agonist to activate 5-LO in granulocytes by a receptor dependent mechanism, thereby providing LTA₄ for conversion to LTC₄ by platelets. As described herein, adherent platelets increased the overall activity of the 5-LO pathway in granulocytes, as reflected by the net quantities of all pathway products detected in the supernatants of the A23187-stimulated samples before and after trypsinization. This priming function could be restored to the trypsinized fractions by adding platelets back (FIG. 8). Activated platelets release large quantities of free arachidonic acid that can induce the translocation and activation of 5-LO in adjacent cells.⁴⁴ Platelets also may prime neutrophils¹⁴ and eosinophils⁴⁵ for augmented 5-LO function by their release of granulocyte macrophage-colony-stimulating factor (GM-CSF).⁴⁶ Thus, platelets probably augment systemic overproduction of cysLTs in AERD by several mechanisms. Urinary concentrations of LTE₄ correlate strongly with certain clinical outcomes in AERD,⁴⁷ and the remarkable correlation between steady-state urinary excretion of LTE₄ and the frequencies of platelet-adherent neutrophils, eosinophils, and monocytes in the peripheral blood (FIGS. 5A-5B), combined with the contribution of platelets to the pool of LTC₄S activity in granulocytes (FIGS. 4A-4D), strongly supports the pathogenetic relevance of the findings described herein. The increases in platelet-leukocyte complexes in AERD are probably not because of enhanced production of thromboxane (FIG. 5A) or isoprostanes, because their respective urinary metabolites (unlike LTE₄ levels) did not discriminate subjects with AERD from aspirin-tolerant controls.

The experiments described herein have identified a disturbance in the homeostasis of interactions between platelets and leukocytes that probably enhances effector cell accumulation in the tissue and augments cysLT production in AERD. Although neither priming for cysLT generation nor priming for leukocyte adhesion are unique functions intrinsic to platelets in AERD, the increased frequencies of platelet-leukocyte aggregates in the blood and nasal polyp tissue of subjects with AERD highlights the potential for a particularly important role in this disease. Given the abundance of neutrophils relative to other leukocyte subsets, the potency of their 5-LO activity, and their tendency to release unmetabolized LTA₄, it is suspected that the ability of platelets to convert neutrophil-derived LTA₄ into LTC₄ may be especially important for controlling basal cysLT levels in AERD. Moreover, the remarkable adherence of platelets to eosinophils suggests that platelets could contribute substantially to the accumulation of eosinophils in the tissue by priming them for adhesion and prolonging their survival (and perhaps reducing their sensitivity to steroid-induced apoptosis) via release of GM-CSF.⁴⁸ Without wishing to be bound by theory, the data described herein indicate that the selective increases in the proportions of eosinophils expressing LTC₄S, in bronchial biopsies⁹ and nasal polyp mucosa¹⁰ obtained from individuals with AERD relative to ATA controls may have been due, in part, to the presence of adherent platelets on the eosinophils.

The inventors' previous work demonstrated that exogenous cysLTs, including LTE₄, do not directly cause human platelets to adhere to granulocytes.⁴⁹ Nonetheless, LTE₄ markedly potentiates pulmonary eosinophilia in mice by a pathway that depends on platelet associated P2Y12 receptors,⁴⁹ the target of thienopyridine drugs. Because thienopyridines also reduce the formation of platelet-leukocyte aggregates,⁵⁰ it is contemplated herein that these antiplatelet therapies could be efficacious as treatments for AERD, both by blocking platelet-leukocyte adhesion and the subsequent formation of cys-LTs and by blocking the actions of LTE₄ by an indirect mechanism.

### REFERENCES

1. Kasper L, Sladek K, Duplaga M, et al. Prevalence of asthma with aspirin hypersensitivity in the adult population of Poland. Allergy 2003; 58(10):1064-1066.
2. Reid G K, Kargman S, Vickers P J, et al. Correlation between expression of 5-lipoxygenase activating protein, 5-lipoxygenase, and cellular leukotriene synthesis. J Biol Chem. 1990; 265(32):19818-19823.
3. Lam B K, Penrose J F, Freeman G J, Austen K F. Expression cloning of a cDNA for human leukotriene C4 synthase, an integral membrane protein conjugating reduced glutathione to leukotriene A4. Proc Natl Acad Sci USA. 1994; 91(16):7663-7667.
4. Christie P E, Tagari P, Ford-Hutchinson A W, et al. Urinary leukotriene E4 concentrations increase after aspirin challenge in aspirin-sensitive asthmatic subjects. Am Rev Respir Dis. 1991; 143(5Pt 1):1025-1029.
5. Drazen J M, Austen K F, Lewis R A, et al. Comparative airway and vascular activities of leukotrienes C-1 and D in vivo and in vitro. Proc Natl Acad Sci USA. 1980; 77(7):4354-4358.
6. Gauvreau G M, Parameswaran K N, Watson R M, O'Byrne P M. Inhaled leukotriene E(4), but not leukotriene D(4), increased airway inflammatory cells in subjects with atopic asthma. Am J Respir Crit Care Med. 2001; 164(8 Pt 1):1495-1500.
7. Dahle'n B, Nizankowska E, Szczeklik A, et al. Benefits from adding the 5-lipoxygenase inhibitor zileuton to conventional therapy in aspirinintolerant asthmatics. Am J Respir Crit Care Med. 1998; 157(4 Pt 1):1187-1194.
8. Stevenson D D, Simon R A, Mathison D A, Christiansen S C. Montelukast is only partially effective in inhibiting aspirin responses in aspirinsensitive asthmatics. Ann Allergy Asthma Immunol. 2000; 85(6 Pt 1):477-482.
9. Cowburn A S, Sladek K, Soja J, et al. Overexpression of leukotriene C4 synthase in bronchial biopsies from patients with aspirin-intolerant asthma. J Clin Invest. 1998; 101(4):834-846.
10. Adamjee J, Suh Y J, Park H S, et al. Expression of 5-lipoxygenase and cyclooxygenase pathway enzymes in nasal polyps of patients with aspirinintolerant asthma. J Pathol. 2006; 209(3):392-399.
11. Sala A, Zarini S, Folco G, Murphy R C, Henson P M. Differential metabolism of exogenous and endogenous arachidonic acid in human neutrophils. J Biol Chem. 1999; 274(40):28264-28269.
12. Penrose J F, Spector J, Lam B K, et al. Purification of human lung leukotriene C4 synthase and preparation of a polyclonal antibody. Am J Respir Crit Care Med. 1995; 152(1):283-289.
13. Maugeri N, Evangelista V, Celardo A, et al. Polymorphonuclear leukocyte-platelet interaction: role of P-selectin in thromboxane B2 and leukotriene C4 cooperative synthesis. Thromb Haemost. 1994; 72(3):450-456.
14. Maclouf J, Antoine C, Henson P M, Murphy R C. Leukotriene C4 formation by transcellular biosynthesis. Ann N Y Acad Sci. 1994; 714:143-750.
15. Bigby T D, Meslier N. Transcellular lipoxygenase metabolism between monocytes and platelets. J Immunol. 1989; 143(6):1948-1954.
16. Pitchford S C, Momi S, Giannini S, et al. Platelet P-selectin is required for pulmonary eosinophil and lymphocyte recruitment in a murine model of allergic inflammation. Blood. 2005; 105(5):2074-2081.
17. Guo Y L, Liu D Q, Bian Z, Zhang C Y, Zen K. Downregulation of platelet surface CD47 expression in Escherichia coli O157:H7 infection-induced thrombocytopenia. PLoS One. 2009; 4(9):e7131.
18. Hsieh F H, Lam B K, Penrose J F, Austen K F, Boyce J A. T helper cell type 2 cytokines coordinately regulate immunoglobulin E-dependent cysteinyl leukotriene production by human cord blood-derived mast cells: profound induction of leukotriene C(4) synthase expression by interleukin 4. J Exp Med. 2001; 193(1):123-133.
19. Borgeat P, Samuelsson B. Metabolism of arachidonic acid in polymorphonuclear leukocytes. Structural analysis of novel hydroxylated compounds. J Biol Chem. 1979; 254(16):7865-7869.
20. Bain G, King C D, Rewolinski M, et al. Pharmacodynamics and pharmacokinetics of AM103, a novel inhibitor of 5-lipoxygenase-activating protein (FLAP). Clin Pharmacol Ther. 87(4):437-444.
21. Morrow J D, Minton T A. Improved assay for the quantification of 11-dehydrothromboxane B2 by gas chromatography-mass spectrometry. J Chromatogr. 1993; 612(2):179-185.
22. Awad J A, Morrow J D, Takahashi K, Roberts L J 2nd. Identification of non-cyclooxygenasederived prostanoid (F2-isoprostane) metabolites in human urine and plasma. J Biol Chem. 1993; 268(6):4161-4169.
23. Morrow J D, Roberts L J, 2nd. Mass spectrometry of prostanoids: F2-isoprostanes produced by non-cyclooxygenase free radical-catalyzed mechanism. Methods Enzymol. 1994; 233:163-274.
24. Massberg S, Brand K, Gruner S, et al. A critical role of platelet adhesion in the initiation of atherosclerotic lesion formation. J Exp Med. 2002; 196(7):887-896.
25. Moritani C, Ishioka S, Haruta Y, Kambe M, Yamakido M. Activation of platelets in bronchial asthma. Chest. 1998; 113(2):452-458.
26. Boilard E, Nigrovic P A, Larabee K, et al. Platelets amplify inflammation in arthritis via collagendependent microparticle production. Science. 2010; 327(5965):580-583.
27. Dees C, Akhmetshina A, Zerr P, et al. Platelet derived serotonin links vascular disease and tissue fibrosis. J Exp Med. 2011; 208(5):961-972.
28. Neumann F J, Marx N, Gawaz M, et al. Induction of cytokine expression in leukocytes by binding of thrombin-stimulated platelets. Circulation. 1997; 95(10):2387-2394.
29. Zarbock A, Singbartl K, Ley K. Complete reversal of acid-induced acute lung injury by blocking of platelet-neutrophil aggregation. J Clin Invest. 2006; 116(12):3211-3219.
30. Jeffery P K, Wardlaw A J, Nelson F C, Collins J V, Kay A B. Bronchial biopsies in asthma. An ultrastructural, quantitative study and correlation with hyperreactivity. Am Rev Respir Dis. 1989; 140(6):1745-1753.
31. Pitchford S C, Riffo-Vasquez Y, Sousa A, et al. Platelets are necessary for airway wall remodeling in a murine model of chronic allergic inflammation. Blood. 2004; 103(2):639-647.
32. Nasser S, Christie P E, Pfister R, et al. Effect of endobronchial aspirin challenge on inflammatory cells in bronchial biopsy samples from aspirinsensitive asthmatic subjects. Thorax. 1996; 51(1):64-70.
33. da Costa Martins P A, van Gils J M, Mol A, Hordijk P L, Zwaginga J J. Platelet binding to monocytes increases the adhesive properties of monocytes by up-regulating the expression and functionality of beta1 and beta2 integrins. J Leukoc Biol. 2006; 79(3):499-507.
34. Xu T, Zhang L, Geng Z H, et al. P-selectin crosslinks PSGL-1 and enhances neutrophil adhesion to fibrinogen and ICAM-1 in a Src kinasedependent, but GPCR-independent mechanism. Cell Adh Migr. 2007; 1(3):115-123.
35. Johansson M W, Mosher D F. Activation of beta1 integrins on blood eosinophils by P-selectin. Am J Respir Cell Mol Biol. 2011; 45(4):889-897.
36. Pretolani M, Ruffle C, Lapa e Silva J R, et al. Antibody to very late activation antigen 4 prevents antigen-induced bronchial hyperreactivity and cellular infiltration in the guinea pig airways. J Exp Med. 1994; 180(3):795-805.
37. Nakajima H, Sano H, Nishimura T, Yoshida S, Iwamoto I. Role of vascular cell adhesion molecule 1/very late activation antigen 4 and intercellular adhesion molecule 1/lymphocyte function associated antigen 1 interactions in antigen induced eosinophil and T cell recruitment into the tissue. J Exp Med. 1994; 179(4):1145-1154.
38. Israel E, Cohn J, Dube L, Drazen J M. Effect of treatment with zileuton, a 5-lipoxygenase inhibitor, in patients with asthma. A randomized controlled trial. Zileuton Clinical Trial Group. JAMA. 1996; 275(12):931-936.
39. Israel E, Chervinsky P S, Friedman B, et al. Effects of montelukast and beclomethasone on airway function and asthma control. J Allergy Clin Immunol. 2002; 110(6):847-854.
40. Fischer A R, Rosenberg M A, Lilly C M, et al. Direct evidence for a role of the mast cell in the nasal response to aspirin in aspirin-sensitive asthma. J Allergy Clin Immunol. 1994; 94(6 Pt 1):1046-1056.
41. Mita H, Higashi N, Taniguchi M, Higashi A, Akiyama K. Increase in urinary leukotriene B4 glucuronide concentration in patients with aspirinintolerant asthma after intravenous aspirin challenge. Clin Exp Allergy. 2004; 34(8):1262-1269.
42. Sala A, Bolla M, Zarini S, Muller-Peddinghaus R, Folco G. Release of leukotriene A4 versus leukotriene B4 from human polymorphonuclear leukocytes. J Biol Chem. 1996; 271(30):17944-8.
43. Fitzpatrick F A, Morton D R, Wynalda M A. Albumin stabilizes leukotriene A4. J Biol Chem. 1982; 257(9):4680-4683.
44. Antoine C, Murphy R C, Henson P M, Maclouf J. Timedependent utilization of platelet arachidonic acid by the neutrophil in formation of 5-lipoxygenase products in platelet-neutrophil co-incubations. Biochim Biophys Acta. 1992; 1128(2-3):139-146.
45. Owen W F, Jr., Rothenberg M E, Silberstein D S, et al. Regulation of human eosinophil viability, density, and function by granulocyte/macrophage colony-stimulating factor in the presence of 3T3 fibroblasts. J Exp Med. 1987; 166(1):129-141.
46. Raiden S, Schettini J, Salamone G, et al. Human platelets produce granulocyte-macrophage colony-stimulating factor and delay eosinophil apoptosis. Lab Invest. 2003; 83(4):589-598.
47. Daffern P J, Muilenburg D, Hugh T E, Stevenson D D. Association of urinary leukotriene E4 excretion during aspirin challenges with severity of respiratory responses. J Allergy Clin Immunol. 1999; 104(3 pt 1):559-564.
48. Her E, Frazer J, Austen K F, Owen W F, Jr. Eosinophil hematopoietins antagonize the programmed cell death of eosinophils. Cytokine and glucocorticoid effects on eosinophils maintained by endothelial cell-conditioned medium. J Clin Invest. 1991; 88(6):1982-1987.
49. Paruchuri S, Tashimo H, Feng C, et al. Leukotriene E4-induced pulmonary inflammation is mediated by the P2Y12 receptor. J Exp Med. 2009; 206(11):2543-2555.
50. Klinkhardt U, Bauersachs R, Adams J, et al. Clopidogrel but not aspirin reduces P-selectin expression and formation of platelet-leukocyte aggregates in patients with atherosclerotic vascular disease. Clin Pharmacol Ther. 2003; 73(3): 232-241.

TABLE 1
Patient Characteristics

| | AERD | ATA | Nonasthmaic |
|---|---|---|---|
| No. | n = 15 | n = 11 | n = 9 |
| Sex (male:female) | 7:8 | 4:9 | 5:4 |
| Median age, y (range) | 45 (20-65) | 37 (22-76) | 34 (22-52) |
| Atopic (n) | 9/15 | 10/13 | 3/9 |
| Baseling FEV 1 (mean % | 82 ± 9 | 88 ± 15 | NA |
| predicted ± SD) | | | |
| Receiving daily inhaled | 15/15 | 12/13 | 0/9 |
| corticosteroids (n) | | | |
| Receiving daily oral | 2/15 | 1/13 | 0/9 |
| corticosteroids (n) | | | |
| Receiving daily long-acting | 10/15 | 7/13 | 0/9 |
| β-agonists (n) | | | |
| Eosinophlis (mean % of blood | 15.1 ± 9.0 | 10.4 ± 6.0 | 6.4 ± 2.7 |
| granulocytes ± SD) | | | |

NA indicates not applicable.

### Example 2

### Adherent Platelets Cause Granulocytes to Secrete More Leukotrienes

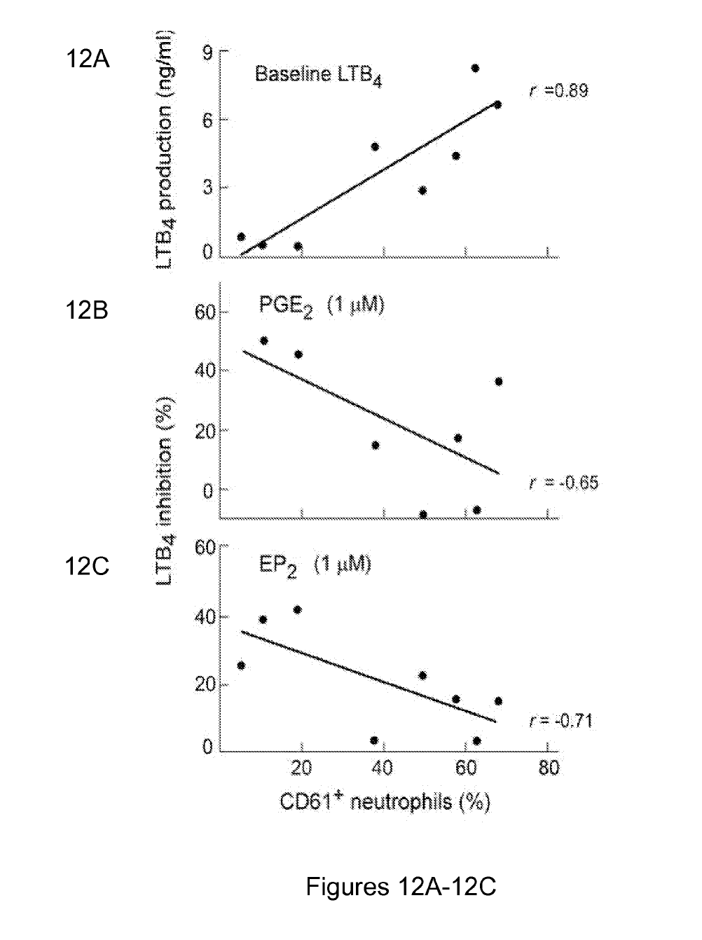

The percentage of platelet-adherent neutrophils (as determined by CD61+ expression) in the whole blood of subjects with AERD was positively correlated with the amount of LTB₄ generated by fMPL-stimulation of granulocytes from the same subjects (FIG. 12A). Treatment with PGE₂ (FIG. 12B) or the EP₂ receptor-specific agonist (FIG. 12C) was able to suppress leukotriene production in these cells. Leukotriene secretion is notable as a signature of aspirin-exacerbated respiratory disease. This data indicates that adherent platelets contribute to the pathophysiology of AERD and validates the use of platelet-bound leukocytes as a marker for AERD.

1.-80. (canceled)

81. An assay comprising:
contacting a test sample comprising leukocytes from the peripheral blood of a subject with:
(a) a platelet-specific antibody reagent selected from the group consisting of a CD61-binding reagent and a CD41-binding reagent; and
(b) a leukocyte-detection antibody reagent selected from the group consisting of a CD45-binding reagent; a CD16-binding reagent; and a CCR3-binding reagent;
detecting the presence or intensity of a detectable signal from the reagents associated with individual cells of the sample using flow cytometry;
calculating the percentage of platelet-bound leukocytes, wherein the binding of platelets to a leukocyte is determined by the colocalization and/or concurrent detection of the platelet-specific antibody reagent and the leukocyte-detecting antibody reagent;
wherein an increased level of platelet-bound leukocytes relative to a reference level indicates the subject has an increased risk of having aspirin-exacerbated respiratory disease (AERD).

82. An assay comprising:
contacting a test sample from a subject with a platelet-specific reagent;
measuring the percentage of leukocytes to which the reagent is bound;
wherein the binding of the platelet-specific reagent to a leukocyte indicates the presence of a platelet bound to the leukocyte;
wherein an increased level of platelet-bound leukocytes relative to a reference level indicates the subject has an increased risk of having aspirin-exacerbated respiratory disease (AERD).

83. The assay of claim 82, further comprising:
(a) contacting the test sample with a platelet-specific antibody reagent and a leukocyte-specific antibody reagent; and
(b) detecting the presence or intensity of a detectable signal associated with individual cells of the sample using flow cytometry;
wherein the antibody reagents comprise a detectable label or a means of generating a detectable signal;
wherein the binding of the platelet-specific antibody reagent to a leukocyte indicates the presence of a platelet-bound leukocyte;
wherein the binding of the platelet-specific antibody reagent to a leukocyte is determined by the colocalization and/or concurrent detection of the platelet-specific antibody reagent and the leukocyte-specific antibody reagent; and
wherein an increased level platelet-bound leukocytes within the population of leukocytes, as indicated by the detectable signals, relative to a reference level indicates the subject has an increased risk of having aspirin-exacerbated respiratory disease (AERD).

84. The assay of claim 82, wherein the platelet-specific reagent is selected from the group consisting of:
a CD61-binding reagent and a CD41-binding reagent.

85. The assay of claim 82, wherein the leukocytes are CD45⁺ cells.

86. The assay of claim 82, wherein the leukocytes are neutrophils.

87. The assay of claim 86, wherein the neutrophils are CD16⁺ cells.

88. The assay of claim 82, wherein the leukocytes are eosinophils.

89. The assay of claim 88, wherein the eosinophils are CCR3⁺ cells.

90. The assay of claim 82, wherein the sample comprises a biological tissue selected from the group consisting of:
whole blood; peripheral blood; whole peripheral blood; a nasal polyp; and products thereof.

91. The assay of claim 82, wherein the subject has increased level of platelet-bound leukocytes relative to a reference level if the level of platelet-bound leukocytes is at least 2× that of the reference level.

92. The assay of claim 82, wherein the subject has increased level of platelet-bound leukocytes relative to a reference level if at least 15% of the leukocytes are platelet-bound leukocytes.

93. The assay of claim 82, wherein the subject has increased level of platelet-bound leukocytes relative to a reference level if at least 50% of the eosinophils are platelet-bound eosinophils.

94. The assay of claim 82, wherein the subject has increased level of platelet-bound leukocytes relative to a reference level if at least 25% of the neutrophils are platelet-bound neutrophils.

95. A method of administering a treatment for a subject with a respiratory disease, the method comprising:
contacting a test sample from the subject with a platelet-specific antibody reagent;
measuring the percentage of leukocytes to which the reagent is bound;
wherein the binding of the reagent to a leukocyte indicates that the leukocyte is a platelet-bound leukocyte; and
administering a therapy for aspirin-exacerbated respiratory disease (AERD) if the level of platelet-bound leukocytes is increased relative to a reference level;
wherein the therapy for AERD is selected from the group consisting of:
aspirin desensitization and high-dose aspirin therapy; a P2Y12 inhibitor; montelukast; a thromboxane receptor antagonist; a 5-lipoxygenase inhibitor; and zileuton;
and not administering a COX1 inhibitor if the level of platelet-bound leukocytes is increased relative to a reference level.

96. The method of claim 95, wherein the platelet-specific reagent is selected from the group consisting of:
a CD61-binding reagent and a CD41-binding reagent.

97. The method of claim 95, wherein the leukocytes are CD45⁺ cells.

98. The method of claim 95, wherein the leukocytes are neutrophils.

99. The method of claim 98, wherein the neutrophils are CD16⁺ cells.

100. The method of claim 95, wherein the leukocytes are eosinophils.

101. The method of claim 100, wherein the eosinophils are CCR3⁺ cells.

102. The method of claim 95, wherein the sample comprises a biological tissue selected from the group consisting of:
whole blood; peripheral blood; whole peripheral blood; a nasal polyp; and products thereof.

103. The method of claim 95, wherein the subject has increased level of platelet-bound leukocytes relative to a reference level if the level of platelet-bound leukocytes is at least 2× that of the reference level.

104. The method of claim 95, wherein the subject has increased level of platelet-bound leukocytes relative to a reference level if at least 15% of the leukocytes are platelet-bound leukocytes.

105. The method of claim 95, wherein the subject has increased level of platelet-bound leukocytes relative to a reference level if at least 50% of the eosinophils are platelet-bound eosinophils.

106. The method of claim 95, wherein the subject has increased level of platelet-bound leukocytes relative to a reference level if at least 25% of the neutrophils are platelet-bound neutrophils.
